(19) United States
(12) Patent Application Publication
(10) Pub. No.: US 2012/0264178 A1
(43) Pub. Date: Oct. 18, 2012

(54) METHODS OF ENABLING ENZYMATIC HYDROLYSIS AND FERMENTATION OF LIGNOCELLULOSIC BIOMASS WITH PRETREATED FEEDSTOCK FOLLOWING HIGH SOLIDS STORAGE IN THE PRESENCE OF ENZYMES

(76) Inventors: Dwight ANDERSON, Puyallup, WA (US); Johnway Gao, Federal Way, WA (US); Benjamin Levie, Mercer Island, WA (US)

(21) Appl. No.: 13/416,911

(22) Filed: Mar. 9, 2012

Related U.S. Application Data
(60) Provisional application No. 61/476,646, filed on Apr. 18, 2011.

(51) Int. Cl. C12P 19/02; C12P 19/00

(52) U.S. Cl. 435/105; 435/72

(57) ABSTRACT

The present invention provides methods of producing pretreated lignocellulosic biomass combined with enzymes for the storage and transporation of the pretreated lignocellulosic biomass that may be used in biofuel and bioproduct production. The methods allows the coexistence of the pretreated lignocellulosic biomass and the enzymes during storage and transporation, the immediate hydrolysis of the pretreated lignocellulosic biomass to produce sugars, without further addition of enzymes, in a biofuel or bioproduct production site, the enhancement of the final hydrolytic activity of the pretreated lignocellulosic biomass, and/or the reduction in sensitivity of the inhibitors in the pretreated lignocellulosic biomass.

### CROSS-REFERENCE TO RELATED APPLICATION

This application claims priority to U.S. Provisional Patent Application Ser. No. 61/476,646, filed Apr. 18, 2011, which is incorporated herein by reference in its entirety.

### FIELD

The present disclosure relates generally to enzymatic hydrolysis of biomass that may be used in biofuel and bioproduct production, and more specifically, to methods of combining pretreated lignocellulosic biomass with hydrolytic cellulase enzymes for the storage and transporation of the pretreated lignocellulosic biomass.

### BACKGROUND

Lignocellulosic biomass is primarily made up of lignin, hemicellulose and cellulose. These three components are tightly bound to each other in the biomass. In order to convert lignocellulosic biomass into a biofuel or a bioproduct, the lignocellulosic biomass has to first be pretreated before enzymatic hydrolysis can take place to produce sugars.

Enzymatic hydrolysis of pretreated lignocellulosic biomass can be done prior to the fermentation of the resulting sugars in a process known as Separate Hydrolysis and Fermentation (SHF), or simultaneously with fermentation in a process known as Simultaneous Saccharification and Fermentation (SSF). In both these processes, the rate of enzymatic hydrolysis affects residence times, which may range from three to five days. The ultimate conversion into a biofuel or a bioproduct can be adversely affected by the presence of inhibitors in the pretreated biomass. These processes are envisioned to be integrated with a pretreatment process that makes the biomass susceptible to enzymatic activity.

Pretreatment of biomass is typically envisioned to occur in the same facility as the conversion to biofuels or bioproducts. In some situations, however, it may be desirable for the pretreatment facility to be located on a different site than the biofuel or bioproduct production facility. In this case, the pretreated biomass would need to be transported from one site to another. In other situations, the pretreatment and production facilities may be on the same site or in close proximity to each other; but the pretreated biomass nonetheless needs to be set aside for several days to weeks before hydrolysis and fermentation will take place in the production facility.

What is needed in the art are methods to produce an intermediate pretreated biomass product that can be set aside, or be transported to a different location until ready for use in enzymatic hydrolysis and conversion into a biofuel or a bioproduct. Commercial equipment is available in the pulp-and-paper industry that makes rolls, slabs, blocks or pellets of cellulosic material for storage or shipping. Such material is routinely stored or shipped at air-dried moisture or at approximately 50% solids as in the case of wet lap. High solids are desirable for the purpose of reducing storage or shipping volume and weight requirements. For example, in U.S. Pat. No. 4,287,823, the slush pulp baler design can achieve 30 lb/cubic foot fiber density. Thus, a significant need exists for methods to produce an intermediate pretreated biomass product that can be stored or shipped in rolls, slabs, blocks or pellets.

### SUMMARY

The present disclosure addresses this need by providing methods to produce pretreated biomass ready for conversion into a biofuel or a bioproduct at a production facility. The methods disclosed herein make it possible to store or transport pretreated biomass that has been somewhat densified by partial dewatering, and has had enzymes applied in a way that can reduce or eliminate the requirement to add enzymes prior to a final conversion process. More specifically, the methods disclosed herein allow (1) the coexistence of the pretreated lignocellulosic biomass and the hydrolytic cellulase enzymes during storage and transporation; (2) the combination of a partial hydrolsis of the pretreated lignocellulosic biomass at a higher density during storage and a more complete hydrolysis upon its dilution to a lower density without further enzyme addition after storage; (3) the immediate hydrolysis of the pretreated lignocellulosic biomass to produce sugars, without further addition of enzymes, in a biofuel or bioproduct production site; (4) the enhancement of the final hydrolytic activity of the pretreated lignocellulosic biomass; and (5) the reduction in sensitivity of the inhibitors in the pretreated lignocellulosic biomass.

One aspect of the disclosure provides a method of preparing pretreated biomass ready for conversion into a biofuel or a bioproduct at a production facility, including the steps of: a) providing biomass; b) applying a treatment method to biomass to produce a pretreated biomass composition that is made up of a pretreatment liquor and pretreated biomass solids; c) separating the pretreatment liquor from the pretreated biomass solids; d) washing the pretreated biomass solids; e) densifying the pretreated biomass solids by removing liquid to form a densified pretreated biomass; f) adding one or more hydrolysis enzymes to the densified pretreated biomass to form a densified enzyme-treated biomass; and g) storing the densified enzyme-treated biomass prior to conversion into a biofuel or a bioproduct at a production facility. In certain embodiments, the method further includes adjusting the pH of the pretreated biomass solids to a pH range of 4.0 to 7.5 after step (d). In some variations, the pH of the pretreated biomass solids is adjusted to a pH range of 4.0 to 6.5. In one variation, the pH of the pretreated biomass solids is adjusted to 5.0. In certain embodiments that may be combined with the preceding embodiments, the treatment method is green liquor, dilute acid, sulfite pulping, bisulfite pulping, kraft pulping, hot water extraction, steam explosion, or a combination of these treatment methods. In certain embodiments that may be combined with the preceding embodiments, the liquid removed in step (e) comprises water, pretreatment liquor, or a mixture thereof. In certain embodiments that may be combined with the preceding embodiments, the densified enzyme-treated biomass is stored at a solids content of 20% to 90%. In one variation, the densified enzyme-treated biomass is stored at a solids content of 30% to 90%, 35% to 80%, or 40% to 70%. In certain embodiments that may be combined with the preceding embodiments, the densified enzyme-treated biomass is stored at a temperature between −30° C. to 50° C. In one variation, the densified enzyme-treated biomass is stored at a temperature between −30° C. to 40° C. In another variation, the densified enzyme-treated biomass is stored at a temperature between 0° C. to 50° C. In yet another variation, the densified enzyme-treated biomass is stored at a temperature between 0° C. to 40° C. In other variations, the densified enzyme-treated biomass is stored at a temperature between 4° C. to 25° C. In yet other variations, the densified enzyme-treated biomass is stored at a temperature between −30° C. and 0° C., or between 30° C. and 50° C. In certain embodiments that may be combined with the preceding embodiments, the one or more hydrolysis enzymes are cellulase, beta-glucosidase, xylanase, other hemicellulases, or a mixture of these hydrolysis enzymes. In certain embodiments that may be combined with the preceding embodiments, the biomass originates from softwood, hardwood, or an herbaceous plant.

Another aspect provides a method of storing pretreated biomass, including the steps of: a) providing biomass; b) applying a treatment method to biomass to produce a pretreated biomass composition that is made up of a pretreatment liquor and pretreated biomass solids; c) densifying the pretreated biomass solids by removing liquid; d) adding one or more hydrolysis enzymes to the pretreated biomass solids to form an enzyme-treated biomass; and e) storing the enzyme-treated biomass at a temperature between −30° C. to 50° C., and at a solids content of 20% to 90%. In certain embodiments, the method further includes adjusting the pH of the pretreated biomass solids to a pH range of 4.0 to 7.5. In some variations, the pH of the pretreated biomass solids is adjusted to a pH range of 4.0 to 6.5. In one variation, the pH of the pretreated biomass solids is adjusted to 5.0. In certain embodiments that may be combined with the preceding embodiments, the treatment method is green liquor, dilute acid, sulfite pulping, bisulfite pulping, kraft pulping, hot water extraction, steam explosion, or a combination of these treatment methods. In certain embodiments that may be combined with the preceding embodiments, the liquid removed in step (c) comprises water, pretreatment liquor, or a mixture thereof. In one variation, the enzyme-treated biomass is stored at a temperature between −30° C. to 40° C. In another variation, the enzyme-treated biomass is stored at a temperature between 0° C. to 50° C. In yet another variation, the enzyme-treated biomass is stored at a temperature between 0° C. to 40° C. In yet another variation, the enzyme-treated biomass is stored at a temperature between 4° C. to 25° C. In yet other variations, the enzyme-treated biomass is stored at a temperature between −30° C. and 0° C., or between 30° C. and 50° C. In certain embodiments, the enzyme-treated biomass is stored at a solids content of 30% to 90%, 35% to 80%, or 40% to 70%. In certain embodiments that may be combined with the preceding embodiments, the one or more hydrolysis enzymes include cellulase, beta-glucosidase, xylanase, other hemicellulases, or a mixture of these hydrolysis enzymes. In certain embodiments that may be combined with the preceding embodiments, the biomass originates from softwood, hardwood, or an herbaceous plant.

Another aspect includes a method of producing pretreated biomass, including the steps of: a) providing biomass; b) applying a treatment method to the biomass to produce a pretreated biomass composition that is made up of a pretreatment liquor and pretreated biomass solids; c) densifying the pretreated biomass solids to a solids content of 20% to 90% by removing liquid; d) adding one or more hydrolysis enzymes to the pretreated biomass solids to form an enzyme-treated biomass; and e) storing the enzyme-treated biomass. In certain embodiments, the method further includes adjusting the pH of the pretreated biomass solids to a pH range of 4.0 to 7.5. In some variations, the pH of the pretreated biomass solids is adjusted to a pH range of 4.0 to 6.5. In one variation, the pH of the pretreated biomass solids is adjusted to 5.0. In certain embodiments that may be combined with the preceding embodiments, the treatment method is green liquor, dilute acid, sulfite pulping, bisulfite pulping, kraft pulping, hot water extraction, steam explosion, or a combination of these treatment methods. In certain embodiments that may be combined with the preceding embodiments, the liquid removed in step (c) comprises water, pretreatment liquor, or a mixture thereof. In certain embodiments that may be combined with the preceding embodiments, the enzyme-treated biomass is stored at a temperature between −30° C. to 50° C. In one variation, the enzyme-treated biomass is stored at a temperature between −30° C. to 40° C. In another variation, the enzyme-treated biomass is stored at a temperature between 0° C. to 50° C. In yet another variation, the enzyme-treated biomass is stored at a temperature between 0° C. to 40° C. In yet another variation, the enzyme-treated biomass is stored at a temperature between 4° C. to 25° C. In yet other variations, the enzyme-treated biomass is stored at a temperature between −30° C. and 0° C., or between 30° C. and 50° C. In some embodiments that may be combined with the preceding embodiments, the densified pretreated biomass is stored at a solids content of 30% to 90%, 35% to 80%, or 40% to 70%. In certain embodiments that may be combined with the preceding embodiments, the one or more hydrolysis enzymes include cellulase, beta-glucosidase, xylanase, other hemicellulases, or a mixture of these hydrolysis enzymes. In certain embodiments that may be combined with the preceding embodiments, the pretreated biomass solids are densified to form a pulp cake, sheet, roll, slab or block. In certain embodiments that may be combined with the preceding embodiments, the biomass originates from softwood, hardwood, or an herbaceous plant.

Another aspect provides a method of producing pretreated biomass, including the steps of: a) providing biomass; b) applying a treatment method to the biomass to produce a pretreated biomass composition that is made up of a pretreatment liquor and pretreated biomass solids; c) separating the pretreatment liquor from the pretreated biomass solids, wherein the pretreated biomass solids have a pH; d) adjusting the pH of the pretreated biomass solids to a pH range of 4.0 to 7.5 to form a pH-adjusted pretreated biomass; e) adding one or more hydrolysis enzymes to the pH-adjusted pretreated biomass solids to form an enzyme-treated biomass; f) densifying the enzyme-treated biomass to a solids content of 20% to 90% by removing liquid to form a densified enzyme-treated biomass; and g) storing the densified enzyme-treated biomass. In certain embodiments, the treatment method is green liquor, dilute acid, sulfite pulping, bisulfite pulping, kraft pulping, hot water extraction, steam explosion, or a combination of these treatment methods. In some variations, the pH of the pretreated biomass solids is adjusted to a pH range of 4.0 to 6.5. In one variation, the pH of the pretreated biomass solids in step (d) is adjusted to 5.0. In certain embodiments that may be combined with the preceding embodiments, the liquid removed in step (f) comprises water, pretreatment liquor, or a mixture thereof. In certain embodiments that may be combined with the preceding embodiments, the densified enzyme-treated biomass is stored at a temperature between −30° C. to 50° C. In one variation, the densified enzyme-treated biomass is stored at a temperature between −30° C. to 40° C. In another variation, the densified enzyme-treated biomass is stored at a temperature between 0° C. to 50° C. In yet another variation, the densified enzyme-treated biomass is stored at a temperature between 0° C. to 40° C. In yet another variation, the densified enzyme-treated biomass is stored at a temperature between 4° C. to 25° C. In yet other variations, the densified enzyme-treated biomass is stored at a temperature between −30° C. and 0° C., or between 30° C. and 50° C. In some embodiments that can be combined with any of the preceding embodiments, the densified enzyme-treated biomass has a solids content of 30% to 90%, 35% to 80%, or 40% to 70%. In certain embodiments that may be combined with the preceding embodiments, the one or more hydrolysis enzymes include cellulase, beta-glucosidase, xylanase, other hemicellulases, or a mixture of these hydrolysis enzymes. In certain embodiments that may be combined with the preceding embodiments, the densified enzyme-treated biomass is in the form of a pulp cake, sheet, roll, slab or block. In certain embodiments that may be combined with the preceding embodiments, the biomass originates from softwood, hardwood, or an herbaceous plant.

Another aspect provides a method of producing pretreated biomass, including the steps of: a) providing biomass; b) applying a treatment method to the biomass to produce a pretreated biomass composition that is made up of a pretreatment liquor and pretreated biomass solids; c) separating the pretreatment liquor from the pretreated biomass solids, wherein the pretreated biomass solids have a pH; d) adjusting the pH of the pretreated biomass solids to a pH range of 4.0 to 7.5 to form pH-adjusted pretreated biomass solids; e) densifying the pH-adjusted pretreated biomass solids by removing liquid to form a densified pretreated biomass that has a solids content of 20% to 90%; f) adding one or more hydrolysis enzymes to the densified pretreated biomass to form a densified enzyme-treated biomass; and g) storing the densified enzyme-treated biomass. In certain embodiments, the method also includes the step of washing the pretreated biomass solids with water before the step of adjusting the pH. The washed pretreated biomass solids may be used in one or more processes where fermenting organisms encounter inhibition from the pretreated biomass solids. In other embodiments, the method also includes the step of mixing the pretreated biomass solids with the pretreatment liquor before the step of adjusting the pH. In yet other embodiments, the pretreated biomass solids are unwashed before the step of adjusting the pH. The unwashed pretreated biomass solids may be used in one or more processes where fermenting organisms can tolerate higher inhibition from the pretreated biomass solids. In some variations, the pH of the pretreated biomass solids is adjusted to a pH range of 4.0 to 6.5. In one variation, the pH of the pretreated biomass solids in step (d) is adjusted to 5.0.

In certain embodiments, the treatment method is green liquor, dilute acid, sulfite pulping, bisulfite pulping, kraft pulping, hot water extraction, steam explosion, or a combination of these treatment methods. In certain embodiments that may be combined with the preceding embodiments, the liquid removed in step (e) comprises water, pretreatment liquor, or a mixture thereof. In certain embodiments that may be combined with the preceding embodiments, the densified enzyme-treated biomass is stored at a temperature between −30° C. to 50° C. In one variation, the densified enzyme-treated biomass is stored at a temperature between −30° C. to 40° C. In another variation, the densified enzyme-treated biomass is stored at a temperature between 0° C. to 50° C. In yet another variation, the densified enzyme-treated biomass is stored at a temperature between 0° C. to 40° C. In yet another variation, the densified enzyme-treated biomass is stored at a temperature between 4° C. to 25° C. In yet other variations, the densified enzyme-treated biomass is stored at a temperature between −30° C. and 0° C., or between 30° C. and 50° C. In some embodiments that may be combined with the preceding embodiments, the densified pretreated biomass has a solids content of 30% to 90%, 35% to 80%, or 40% to 70%.

In certain embodiments that may be combined with the preceding embodiments, the densified enzyme-treated biomass is in the form of a pulp cake, sheet, roll, slab or block. The densified enzyme-treated biomass after storage may be diluted to 5% to 30% solids content prior to hydrolysis under suitable conditions to produce monomer sugars, where the hydrolysis produces a glucose yield of 70% to 100% of the pretreated biomass composition. The sugars produced by the hydrolysis may be fermented with one or more fermentation organisms to produce a fermentation product, where the fermentation converts 60% to 100% of the sugars to the fermentation product. In some embodiments, the fermentation product may include alcohols, organic acids, amino acids, diols, proteins, gases, and lipids. The alcohols may include, for example, ethanol, butanol, and isobutanol. The organic acids may include, for example, acetic acid, lactic acid, and citric acid. The amino acids may include, for example, lysine, methionine, alanine, and glutamic acid. The diols may include, for example, propanediol and butanediol. The proteins may include, for example, enzymes and polypeptides. The gases may include, for example, biogas, methane, hydrogen and carbon dioxide. Fermenting organisms may include yeast, fungi, mold, algae, bacteria, or a mixture of these fermenting organisms. For example, in some embodiments, the fermenting organisms may be Escherichia coli or Clostridium. In other embodiments, the fermenting organisms may be genetically modified, altered or engineered.

In certain embodiments, the pretreatment liquor may be used for biofuel or bioproduct production. In certain embodiments, the pretreatment liquor may be used for biogas production. In certain embodiments, the pretreatment liquor may be used for lignosulfonate production. In certain embodiments, the biogas production produces one or more products that may include alcohols (e.g., ethanol, butanol, and isobutanol), organic acids (e.g., acetic acid, lactic acid, and citric acid), amino acids (e.g., lysine, methionine, alanine, and glutamic acid), diols (e.g., propanediol and butanediol), proteins (e.g., enzymes and polypeptides), gases (e.g., biogas, methane, hydrogen and carbon dioxide), and lipids.

In certain embodiments that may be combined with the preceding embodiments, the method also includes adding one or more hydrolysis enzymes to the densified enzyme-treated biomass after storage. In certain embodiments that may be combined with the preceding embodiments, the one or more hydrolysis enzymes include cellulase, beta-glucosidase, xylanase, other hemicellulases, or a mixture of these enzymes. In certain embodiments that may be combined with the preceding embodiments, the one or more hydrolysis enzymes are uniformly added to the pretreated biomass solids. In one variation, the one or more hydrolysis enzymes are sprayed on the pretreated biomass solids. In another variation, the one or more hydrolysis enzymes are added uniformly to the sheet of pretreated biomass. In yet another variation, the one or more hydrolysis enzymes are sprayed on the sheet of pretreated biomass. In some variations, the one or more hydrolysis enzymes are added in combination with the use of a slush pulp packaging, and the one or more hydrolysis enzymes are uniformly distributed within the slab or block of pretreated biomass. In certain embodiments that may be combined with the preceding embodiments, the sheets, rolls, slabs or blocks are produced in a general clean-in-place process. In certain embodiments that may be combined with the preceding embodiments, the biomass originates from softwood, hardwood, or an herbaceous plant.

### BRIEF DESCRIPTION OF THE DRAWINGS

The present application can be best understood by reference to the following description taken in conjunction with the accompanying figures.

FIG. 1. Glucose and ethanol titer of hydrolysis and fermentation after 1-week incubation of pulp cakes with initial 100%, 20% and 0% of enzyme.

FIG. 2. Glucose and ethanol titer of hydrolysis and fermentation after 2-week incubation of pulp cakes with initial 100%, 20% and 0% of enzyme.

FIG. 3. Process flow diagram for pretreated pulp solid washing and pulp cake production without enzyme addition to pulp cake.

FIG. 4. Process flow diagram for pretreated pulp solid washing and pulp cake production with enzyme addition to pulp cake.

FIG. 5. Process flow diagram for pretreated pulp cake production without enzyme addition to pulp cake.

FIG. 6. Process flow diagram for pretreated pulp cake production with enzyme addition to pulp cake.

### DETAILED DESCRIPTION

The following description sets forth exemplary methods, parameters and the like. It should be recognized, however, that such description is not intended as a limitation on the scope of the present invention but is instead provided as a description of exemplary embodiments. From these, a person of ordinary skill would be able to practice the invention without undue experimentation.

### 1. Definitions

As used herein, “biomass sizing” refers to reducing the size of the wood chip in a pretreatment process to enable less severity of time or temperature. For woody feedstock in particular, biomass sizing is an effective practice for reducing inhibitors. Biomass sizing may reduce any conditioning requirement of the liquid prehydrolysate, better enabling it to serve as a diluent for enzymatic hydrolysis

As used herein, “treatment method” or “pretreatment” refers to a method of using mechanical, chemical, thermal and/or enzymatic hydrolytic method(s) to make cellulose and/or hemicellulose available for a chemical and/or an efficient enzymatic hydrolysis of lignocellulosic biomass or materials to produce monomeric sugars. Unless indicated otherwise, a treatment method does not include further processing steps such as separation of solid and liquid phases of the pretreatment product or rinsing or conditioning of the solid or liquid product phases.

As used herein, “pretreatment liquor” or “prehydrolysate” refers to a liquid fraction of the pretreatment reaction mixture.

As used herein, “pretreated biomass solids” refer to biomass solids that have undergone pretreatment, and unless otherwise indicated, a pretreated biomass solid has not received other treatments or processing.

As used herein, “solids content” refers to the amount of material left in the biomass after water or liquor removal, and is expressed as a percentage by weight.

As used herein, “pulp cake”, “sheet”, “roll”, “slab” and “block” refer to pretreated pulp materials that are dewatered and densified. For example, pretreated pulp materials could be dewatered to form a cake or sheet by filtration or compression after pH adjustment. The cake or sheet could be subsequently stacked up to form a thick pulp slab, or a block of multi layers of pulp cake, sheet or roll.

As used herein, “clean packaging” refers to a packaging method that minimizes or eliminates unwanted contaminants in the packaged pretreated-lignocellulosic biomass or materials. The contaminants include unwanted microorganisms and chemicals that will cause the pretreated biomass to rot or become inhibitive to subsequent processing.

As used herein, “enzymatic hydrolysis” or “enzymatic hydrolysis of pretreated biomass” refers to the hydrolytic process of a pretreated biomass by one or more enzymes or cellulases to produce oligomer and/or monomeric sugars.

As used herein, “fermentation organisms” refer to microorganisms that can convert a substrate or sugar(s) in fermentation process to produce a product. Examples of these organisms include mold, yeast, algae, and bacteria.

As used herein, when the term “about” modifies a number, the term is defined as “approximately,” and the number should be interpreted to cover a range that includes its recited value and the experimental error in obtaining the number.

Terms and phrases used in this document, and variations thereof, unless otherwise expressly stated, should be construed as open ended as opposed to limiting. As examples of the foregoing: the term “including” should be read to mean “including, without limitation” or the like; the terms “example” or “some variations” are used to provide exemplary instances of the item in discussion, not an exhaustive or limiting list thereof; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of methods and compositions described herein may be described or claimed in the singular, the plural is contemplated to be within the scope thereof unless limitation to the singular is explicitly stated. The presence of broadening words and phrases such as “one or more,” “at least,” “but not limited to,” “in some variations,” “in some non-limiting variations” or other like phrases in some instances shall not be read to mean that the narrower case is intended or required in instances where such broadening phrases may be absent.

### 2. Description

The present disclosure provides a method of producing lignocellulosic biomass between 20% and 90% solids content that has been treated to facilitate conversion to a biofuel or a bioproduct, and includes the application of an enzyme or enzyme cocktail to pretreated biomass that is stored at conditions outside the optimal range of solids and temperature for conversion.

### Biomass

Biomass is plant material that is made up of organic compounds relatively high in oxygen, such as carbohydrates, and may also contain a wide variety of other organic compounds. Lignocellulosic biomass is a type of biomass that is made up of cellulose and hemicellulose bonded to lignin in plant cell walls. Lignocellulosic biomass can be grouped into four main categories: agricultural residues (e.g. corn stover, sugarcane bagasse), dedicated energy crops (e.g. sugarcane), wood residues (e.g. sawmill, paper mill discards, softwood chips, hardwood chips), and municipal paper waste. Any source of biomass can be used in these methods, and some typical examples are described herein. Lignocellulosic biomass may originate from softwood, hardwood, or an herbaceous plant. Wood chips and bark materials from these sources can be used as a suitable biomass for the methods described herein.

### Treatment Methods

Digestibility of cellulose in lignocellulosic biomass is hindered by various physicochemical, structure and compositional factors. As such, treatment of lignocellulosic biomass is needed to facilitate enzymatic hydrolysis for sugar production. Treatment of lignocellulosic biomass will expose the cellulose in the plant fibers by breaking down the lignin structure and disrupting the crystalline structure of cellulose, thereby making the biomass more accessible to enzymatic hydrolysis. Treatment methods may be physical, chemical, physicochemical or biological, or involve a combination of these treatment methods.

Physical treatment methods often involve size reduction to reduce the physical size of biomass. Numerous physical treatment methods are known in the art. Examples include chipping, grinding, shredding, chopping, milling, and pyrolysis.

Chemical treatment methods often involve removing chemical barriers to allow enzymes to access the cellulose for microbial destruction. Numerous chemical treatment methods are known in the art. Examples include acid hydrolysis, alkaline hydrolysis, ozonolysis, oxidative delignification, organic solvents, ionic liquids (IL), electrolyzed water, sulfite or bisulfite pulping, kraft pulping, and green liquor.

One skilled in the art is aware of numerous physicochemical treatment methods. Examples include steam explosion with or without sulfur dioxide, ammonia fiber explosion (AFEX), and carbon dioxide explosion. One skilled in the art is also aware of numerous biological treatment methods. Examples include various types of rot fungi (e.g. brown-, white-, and soft-rot fungi). Examples of other treatment methods include pulsed-electric-field pretreatment (PEF).

Applying some of the treatment methods described above to lignocellulosic biomass produces a pretreatment biomass composition, which can be separated into pretreatment liquor and pretreated biomass solids.

a) Pretreatment Liquor

Pretreatment liquor, also known as prehydrolysate, is a liquid fraction, which is typically rich in hemicellulose sugars or hemicellulose oligomers, along with lignin, extractives, furans, aldehydes, acetic acid, or other inhibitors that restrict the growth and productivity of a fermenting organism. Pretreatment liquor usually has a pH range that is outside of the typical enzymatic hydrolysis pH range or typical fermentation pH range. Moreover, pretreatment liquor may be used in a separate process for biofuel production, bioproduct production, or biogas production.

b) Pretreated Biomass Solids

Pretreated biomass solids are a solid fraction, which is typically rich in cellulose. Similar to the pretreatment liquor, pretreated biomass solids also may also contain inhibitors and a different pH from the enzymatic hydrolysis pH and the fermentation pH. Therefore, pretreated biomass solids often need to be conditioned before an enzymatic hydrolysis and a fermentation process.

As part of the conditioning before hydrolysis and fermentation, pretreated biomass solids are typically washed to remove fermentation inhibitors. Washing helps promote safer material storage and transportation, as well as helps maintain enzyme activity during storage. Pretreated biomass solids may be washed with water. If pretreated biomass solids are not washed, pretreated biomass solids may be mixed with the pretreatment liquor for safer material storage and transportation, as well as for maintaining enzyme activity during storage. In other situations, pretreated biomass solids are unwashed.

The pH of pretreated biomass solids is typically low or high. As a result, the pH of pretreated biomass solids needs to be adjusted as part of the conditioning before enzymatic hydrolysis. One skilled in the art would recognize various techniques that can be used to adjust the pH to a suitable condition for enzymatic hydrolysis. Examples include the use of buffers. The pH of pretreated biomass solids may be adjusted to a range of 4-7.5, or a range of 4-6.5, or preferably 5.0.

### Densification

In order to ship or transport pretreated biomass, pretreated biomass solids are typically densified in the form of rolls, slabs, blocks or pellets. Densification is a process of making biomass more compact by increasing the mass per unit of volume. Densification presents the advantage of making handling, storage and transportation of biomass easier and less expensive. Cost savings can be realized when biomass is densified because, for example, fewer silos are needed for storage and fewer trucks are needed for transportation.

Various methods for biomass densification are known in the art. Examples include extrusion, briquetting, pelletizing, compaction, filtration, and compression. Biomass may also be densified by removing water, the pretreatment liquor or a mixture thereof. The water and/or pretreatment liquor are removed from the pretreated biomass solids by filtration or compression to form a pulp cake, a sheet, or a roll. This cake or sheet can then be stacked to form a pulp slab, or a block made up of multi-layers of pulp cake, sheet, or roll.

In producing the pulp slabs or blocks, a general clean-in-place process is needed to ensure that lignocellulosic biomass, the hydrolyzing process, and the fermenting process, or the combination of such processes are free of contaminating organisms that may significantly affect biofuel or bioproduct production.

### Enzyme Application

The present disclosure teaches methods of producing pretreated biomass that is ready for conversion into a biofuel or a bioproduct at a production facility. In order for pretreated biomass to be ready for conversion after taken out of storage or upon delivery to the production facility, one or more hydrolysis enzymes are applied to pretreated biomass.

a) Hydrolysis Enzymes

Hydrolysis enzymes catalyze the conversion of biomass into monomeric and/or oligomeric sugars. One skilled in the art is aware of various hydrolysis enzymes. Examples include cellulases, beta-glucosidases, xylanases, endoxylanases,β-xylosidases, β-glucosidases, arabinofuranosidases, glucuronidases, and acetyl xylan esterases. Combinations of enzymes (i.e. enzyme cocktails) can also be tailored to the structure of a specific biomass feedstock to increase the level of degradation.

b) Timing

One or more hydrolysis enzymes may be applied to pretreated biomass solids after densification. If applied after a pulp cake or sheet is formed, a concentrated enzyme is sprayed or spread onto the pulp cake or sheet. One or more enzymes may also be applied to pretreated biomass before densification. If applied before densification, the pressing of the pulp may release prehydrolysate that contains sugars and enzymes.

c) Enzyme Dosing

In applying one or more hydrolysis enzymes to pretreated biomass, various doses may be used. In one variation, 100% of the enzymes needed for hydrolysis may be applied before storage. In another variation, 20% of the enzymes needed for hydrolysis may be applied before storage, and the remaining 80% of the enzymes are applied after storage.

d) Application Methods

In one variation, one or more hydrolysis enzymes are applied to pretreated biomass in a way that results in a roughly uniform distribution of enzymes. When enzymes are applied to a densified and dewatered sheet of pretreated lignocellulosic biomass, the one or more hydrolysis enzymes are applied to achieve a roughly uniform distribution of enzymes in the two dimensional-plane of the sheet. When enzymes are applied to a pulp slab or block, the one or more hydrolysis enzymes are applied to achieve a roughly uniform distribution of enzymes within the three dimensions of the pulp slab or block.

In some variations, one or more hydrolysis enzymes may be sprayed onto the pretreated biomass to achieve uniform application. The methods described in U.S. application Ser. No. 12/816999 (filed Jun. 16, 2010) may be used to spray one or more hydrolysis enzymes onto pretreated biomass. In other variations, one or more hydrolysis enzymes may be applied to pretreated biomass in a mixing tank, following by pressing and/or drying.

Storage and/or Transportation

Pretreated biomass that has been densified into a pulp cake, sheet, roll, slab or block can be stored or transported from a pretreatment facility to a production facility. As discussed above, a high solids content is desirable for the purpose of reducing storage or shipping volume and weight requirements. The solids content of biomass during storage may be 20-90%, 20-80%, 20-70%, 20-60%, 20-50%, 20-40%, 20-30%, 30-90%, 30-80%, 30-70%, 30-60%, 30-50%, 30-40%, 35% to 80%, or 40% to 70%. One of skill in the art would recognize, however, that enzymatic activity is low at high solids content, e.g., above about 20-30%.

Storage at unregulated temperatures is also desirable so as to reduce costs from regulating the conditions in a storage facility, and to transport pretreated biomass between a pretreatment facility to a production facility. One of skill in the art would recognize, however, that freezing or storing the enzymes above ambient temperatures could lead to reduction or loss of enzymatic activity. For example, when enzymes are stored at temperatures below 0° C. or above 30° C., the stability of the enzymes may be affected and enzymatic activity may be lost. Smith et al. have found that the hydrolytic efficiency of enzymes stored for 10 days at 45° C. was only 60% of the efficiency of fresh enzyme after 24 hours of hydrolysis. See Smith, B. T., J. S. Knutsen, and R. H. Davis, “Empirical Evaluation of Inhibitory Product, Substrate, and Enzyme Effects During the Enzymatic Saccharification of Lignocellulosic Biomass,” Applied Biochemistry and Biotechnology 161, 468-482 (2010).

Storage according to the methods described herein may be at a temperature near ambient conditions but below 50° C. In one variation, storage may be at a temperature between −30° C. to 50° C. In another variation, storage may be at a temperature between −20° C. to 50° C. In yet another variation, storage may be at a temperature between 0° C. to 50° C. In yet another variation, storage may be at a temperature between 0° C. to 40° C. In other variations, storage may be at a temperature between 4° C. to 25° C. In some variations, storage may be at a temperature between 25° C. to 40° C. In yet other variations, storage may be at a temperature between 15° C. to 25° C. In yet other variations, storage may be at a temperature between 20° C. to 25° C. In yet other variations, storage may be at a temperature between −30° C. and 0° C., or between 30° C. and 50° C.

Storage may also be at any humidity up to 100% relative humidity. Depending on the reason for storage or the distance between pretreatment facility and production facility, storage may be for a period of at least one week. In one variation, storage may be for one day, several days, one week, several weeks, one month, or several months.

### Hydrolysis

Pretreated biomass is hydrolyzed under suitable conditions to produce sugars. Much is known about factors that relate to enzymatic hydrolysis. Hydrolysis rates increase with temperature, but at too high a temperature the enzymes will become denatured. High solids are desirable for high titer, but the percentage of theoretical hydrolysis achieved decreases with increased solids. Kristensen et al. hypothesized that this was due to inhibition by the products of hydrolysis. See Jan B. Kristensen, et al., Yield-determining factors in high solids enzymatic hydrolysis of lignocellulose, Biotechnology for Biofuels 2, 11 (2009). This effect is strong enough to make 20% solids a practical upper limit for enzymatic hydrolysis. Moreover, the addition of enzymes above 20% solids in an integrated process is not expected to have the same level of hydrolytic performance as a process at a lower consistency, such as 15%.

The methods and conditions suitable for enzymatic hydrolysis to convert lignocellulosic biomass into sugars are well known in the art. For example, Tengborg et al. teach one way for enzymatic hydrolysis of steam-pretreated softwood, such as spruce, for sugar production. See Charlotte Tengborg, et al., Influence of enzyme loading and physical parameters on the enzymatic hydrolysis of steam pretreated softwood, Biotechnol. Prog. 17: 110-117 (2001).

After removal from storage, the densified biomass is diluted to 5% to 30% solids content before hydrolysis. In another variation, the densified biomass is diluted to 5% to 20% solids content prior to hydrolysis. In yet another variation, the densified biomass is diluted to 5% to 15% solids content prior to hydrolysis. In yet another variation, the densified biomass is diluted to 5% to 10% solids content prior to hydrolysis. In yet another variation, the densified biomass is diluted to 5% solids content prior to hydrolysis.

### Fermentation

Hydrolyzed or semi-hydrolyzed lignocellulosic materials are fermented with one or more fermenting organisms to produce a fermentation product. The fermentation product may be a biofuel (e.g. ethanol, propanol, butanol, etc.) or a bioproduct (e.g. amino acids, organic acids, pharmaceuticals, specialty chemicals etc.). The fermentation process may use fermentation organisms such as yeast, fungi, mold, algae, bacteria, or a mixture of these organisms. Fermentation organisms may also include Escherichia coli and Clostridium.

The methods and conditions suitable for sugar fermentation into a biofuel or a bioproduct are well known in the art. For example, Sedlak and Ho teach one way to produce ethanol from sugar fermentation of cellulosic biomass, such as corn stover. See Miroslav Sedlak and Nancy W. Y. Ho, Production of ethanol from cellulosic biomass hydrolysates using genetically engineered Saccharomyces yeast capable of cofermenting glucose and xylose, Applied Biochemistry and Biotechnology, 113-116: 403-416 (2004).

In some variations, fermentation conditions are maintained for 24 hours to 72 hours. In some variations, fermentation conditions are maintained for 36 hours to 60 hours. In some variations, fermentation converts 60% to 100% of the sugars to the fermentation product.

Although individual features of the methods described herein may be included in different claims, these may be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate. Where a composition or process ‘comprises’ one or more specified items or steps, others can also be included. The invention also contemplates, however, that the described composition or process may be used without other items or steps and thus it includes the recited composition or process ‘consisting of’ or ‘consisting essentially of’ the recited items, materials or steps, as those terms are commonly understood in patent law.

### EXAMPLES

The following Examples are merely illustrative and are not meant to limit any aspects of the present disclosure in any way.

### Reagents

Calcium bisulfite was produced by constantly purging pure sulfur dioxide to a calcium oxide solution. The final calcium bisulfite concentration contains about 3-4% total sulfur dioxide (combined plus free), of which about 1% is free sulfur dioxide. The pH of this calcium bisulfite solution is around 1.4. The free sulfur dioxide in solution is also called a sulfurous acid solution. This acid calcium bisulfite solution is widely used in an acid sulfite pulping process in the pulp and paper industry.

Cellulase (Celluclast, Sigma Catalog #C-2730), Cellic® CTec2 enzyme product (Novozymes), beta-glucosidase (Novozymes-188, Sigma Catalog #C-6105), and xylanase (Novozyme NS50030) were used, accordingly in the enzymatic hydrolysis experiments after the pretreatment to determine the glucose yield from the pretreated materials and in the pulp storage tests. A yeast strain Saccharomyces cerevisiae T2 was obtained from Dr. Sheldon Duff at the University of British Columbia. This yeast strain was used for ethanol fermentation after the pulp hydrolysis process.

Forest Residual Pretreatment with a Conventional Chip Digestion Method by Calcium Bisulfite

Forestry residual materials containing both wood chips and bark materials were obtained from a pulp mill in the southern United States. It should be recognized, however, that the biomass feedstocks used in the methods described herein may be softwood forest residuals, hardwoods (e.g., maple hardwood chips), switchgrass, or any lignocellulosic feedstock. The softwood forestry residual materials and maple hardwood chips were pretreated before an enzymatic hydrolysis for sugar production. Before pretreatment, the softwood forestry residuals and hardwood chips were further fractured with a BearCat garden chipper with a ¾″ screen to obtain the “re-chipped” materials. For the re-chipped chips, the 3-mm round hole fines were removed to avoid circulation problems in the lab pretreatment reactor.

The re-chipped chips were pretreated in a one cubic foot reactor with an acid sulfite pretreatment consisting of 12.5% calcium bisulfite on wood with a single step temperature schedule: ramped from 90° C. to 155° C. in 15 minutes and held at 155° C. for 120 minutes. After cooking, the liquor was drained and the cooked chips were collected. The cooked chips were then sent to an Alpine grinder, without any water, to refine the chips into a pulp. The pulp batch number for this cook was CS 10219A and this pulp was used in the following unwashed pulp test. Another 12.5% calcium bisulfite cook had a single step but a different temperature: ramped from 90° C. to 165° C. in 15 minutes and held at 165° C. for 75 minutes. The pulp batch number for this cook was CS10221A and this pulp was used in the following washed pulp test. The pretreatment temperature at 165° C. was close to 160° C. to 170° C. that were reported in some acid sulfite pulp processes by Seaman in 1954 (U.S. Pat. No. 2,698,234) and by Wolfinger et al. in 2004 (Martin G. Wolfinger & Herbert Sixta, Modeling of the acid sulfite pulping process.—Problem definition and theoretical approach for a solution with the main focus on the recovery of cooking chemicals, Lenzinger Berichte, 83: 35-45 (2004)).

In pretreatment, the solubilization of woody materials was approximately 25% on a dry wood basis. The prehydrolysate or the pretreatment liquor was collected. The pretreated and unwashed solids were ground into fine pulp in an Alpine grinder without any dilution water. The ground solids were subjected to enzymatic hydrolysis at 5% solids in a 50 mmol pH 4.8 citrate buffer with 0.27 g Celluclast enzyme product/dry gram of pretreated solids, 0.080 gram Novozyme-188 beta-glucosidase/dry gram of pretreated solids and 0.016 gram xylanase product/dry grams of pretreated solids. After 48 hours of enzymatic hydrolysis, the total sugar conversion yield from the pretreated materials was 90.4%, on the basis of total dry and un-pretreated woody materials.

### Washed Pretreated Cellulosic Material Preparation

The ground pretreated pulp had a pH of about 1.4. For safer material storage and transportation and for maintaining enzyme activity during pulp storage, the ground pulp materials were washed in 4x water and the pH was adjusted to about 4.5 to 5.0 with calcium oxide. Subsequently, the washed pulp was filtered in a vacuum filter. The filtered pulp was formed into a cake or a thick sheet on the filtration unit and was further pressed to remove excessive water to achieve a solid content of about 22%. The cake thickness was about 1 centimeter.

For testing, a 30-gram pulp cake was transferred into a 125 ml flat bottom Erlenmeyer flask. A spatula was used to tap the pulp cake tightly onto the flask bottom. The pulp cake in the flask was sterilized at 250° F. for 20 minutes. After cooling down to room temperature, one set of pulp cakes were applied with Cellic® CTec2 enzyme at a dose of 0.14 gram enzyme product (nominal 100% enzyme)/dry gram of pulp materials, and the other set of pulp cakes were applied with Cellic® CTec2 enzyme at a dose of 0.028 gram enzyme product (nominal 20% enzyme)/dry gram of pulp. The enzymes were only applied to the top of the pulp cake. The enzymes were applied evenly using a pipette, and no mixing was used. No enzymes were applied to the control set. After this procedure, each flask mouth was wrapped tightly with two layers of aluminum foil, and placed into a plastic tub that was wrapped with several layers of plastic wraps to avoid any moisture lost during storage. The tub with all the flasks was stored in an environmental chamber with a set temperature of 23° C. and a set humidity of 20%. Three different sets of flasks were taken out at T=0, 1, 2 and 4 weeks for enzymatic hydrolysis and fermentation tests.

### Example 1

### Hydrolysis and Fermentation of Washed Pretreated Softwood Cellulosic Cake Hydrolysis and Fermentation

At T=0 weeks (i.e. no storage), a set of the washed pulp cakes applied with 0.14 and 0.028 gram enzyme product/dry gram of pulp materials was taken out. More enzymes were added to the pulp cakes with 0.028 and 0.0 gram enzyme product/dry gram of pulp materials so that the total enzyme dose was 0.14 gram enzyme product/dry gram of pulp materials. After enzyme addition, a 50 mmol sodium citrate buffer (pH 4.8) was added to the pulp materials, and mixed using a spatula. The flasks were incubated in a shaking incubator at 50° C. and 200 rpm. After about 2 days of enzymatic hydrolysis, yeast seed was added to each flask at about 2 g/L for ethanol fermentation. The fermentation temperature was controlled at 38° C., and the mixing was controlled at 100 rpm for flask mixing. The final pulp consistency in fermentation was 15.7%.

The normalized glucose yield was calculated by dividing the total amount of glucose released during a hydrolysis by the maximum amount of glucose in a control test with sufficient amount of enzyme for complete glucan hydrolysis. The ethanol yield was calculated by dividing the weight percent of ethanol produced in fermentation by the total initial weight percent sugar in the fermentation mixture from the added pulp sample, then dividing by its sugar-to-ethanol theoretical yield. Since the yeast in this Example only used C6 sugars (e.g., glucose) but not C5 sugars (e.g., xylose and arabinose), the sugar-to-ethanol theoretical yield used for the calculation was 0.511 g of ethanol/g glucose.

The glucose yields of enzymatic hydrolysis at 51 hrs and ethanol titers at 75 and 99 hrs were determined, as shown below in Table 1. The test results showed that most of the glucan was hydrolyzed at a yield of about 89-93%. The ethanol fermentation yield to total pulp sugar was about 80-83%.

TABLE 1
Glucose yields and ethanol fermentation yields in pulp hydrolysis
and fermentation after T = 0 week pulp cake incubation with enzyme

| | | | | | | |
|---|---|---|---|---|---|---|
| | | | Normalized | | | Maximum |
| | | | Glucose | Ethanol | Ethanol | Ethanol |
| | | Glucose | Yield (%) | Titer | Titer | Yield (%) |
| | | (%) at | on Pulp | (%) at | (%) at | on Pulp |
| Week 0 | Test No. | 51 hr | Glucose | 75 hr | 99 hr | Sugar |

Pulp Cake Stored with 100% Enzyme Initially

| | | | | | |
|---|---|---|---|---|---|
| 100W1 | 9.4 | 88.9 | 3.7 | 3.7 | 79.7 |
| 100W2 | 9.9 | 93.2 | 3.9 | 3.9 | 83.3 |

Pulp Cake Stored with 20% Enzyme Initially + 80% Enzyme at Start of Conversion

| | | | | | |
|---|---|---|---|---|---|
| 20W1 | 9.4 | 88.8 | 3.7 | 3.7 | 79.3 |
| 20W2 | 9.5 | 90.0 | 3.8 | 3.7 | 79.2 |

### Example 2

### Washed Pretreated Softwood Cellulosic Cake 1-Week Storage with Enzyme

At T=1 week storage, a set of the washed pulp cakes applied with both 0.14 and 0.028 gram enzyme product/dry gram of pulp materials was taken out. More enzymes were added to the pulp cakes with 0.028 and 0.0 gram enzyme product/dry gram of pulp materials so that the total enzyme dose was 0.14 gram enzyme product/dry gram of pulp materials. After enzyme addition, a 50 mmol sodium citrate buffer (pH 4.8) was added to the pulp materials, and mixed well using a spatula. The flasks were incubated in a shaking incubator at 50° C. and 200 rpm. After about 2 days of enzymatic hydrolysis, yeast seed was added to each flask at about 2 g/L for ethanol fermentation. The fermentation temperature was controlled at 38° C., and the mixing was controlled at 100 rpm for flask mixing. The final pulp consistency in fermentation was 15.7%.

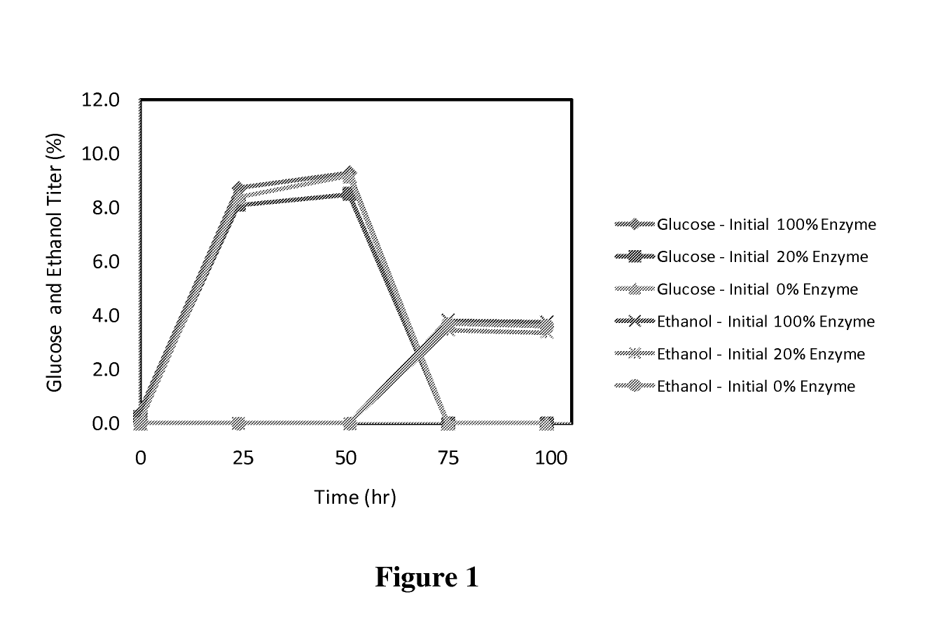

FIG. 1 shows the plot of glucose titers and ethanol concentrations during the course of hydrolysis and fermentation. At about 24 hours, most of the hydrolysis was observed to be completed. At 51 hours, a yeast seed was added, after which ethanol fermentation was observed to be mostly completed in 24 hours. The actual time for hydrolysis and fermentation was observed to be as short as 48 hours.

The glucose yields of enzymatic hydrolysis at 51 hrs and ethanol titers at 75 and 99 hrs for the week 1 stored pulp cakes were determined, as shown below in Table 2. The test results showed that most of the glucan was hydrolyzed at glucose yields of about 88% and about 80%, respectively for the initial 100% enzyme added pulp cake tests and for the initial 20% enzyme added pulp cake tests. The ethanol fermentation yields to total pulp sugar were about 82% and about 74%, respectively for the initial 100% and 20% enzyme added cake tests. The control test had about 86% glucose yield and about 79% ethanol fermentation yield.

TABLE 2
Glucose yields and ethanol fermentation yields in pulp hydrolysis and
fermentation after T = 1 week pulp cake incubation with enzyme

| | | | | | | |
|---|---|---|---|---|---|---|
| | | | Normalized | | | Maximum |
| | | | Glucose | Ethanol | Ethanol | Ethanol |
| | | Glucose | Yield (%) | Titer | Titer | Yield (%) |
| | | (%) at | on Pulp | (%) at | (%) at | on Pulp |
| Week 1 | Test No. | 51 hr | Glucose | 75 hr | 99 hr | Sugar |

Pulp Cake Stored with 100% Enzyme Initially

| | | | | | |
|---|---|---|---|---|---|
| 100W3 | 9.3 | 87.4 | 3.8 | 3.9 | 83.3 |
| 100W4 | 9.3 | 88.0 | 3.8 | 3.7 | 80.5 |

Pulp Cake Stored with 20% Enzyme Initially + 80% Enzyme at Start of Conversion

| | | | | | |
|---|---|---|---|---|---|
| 20W3 | 8.6 | 80.8 | 3.6 | 3.5 | 76.0 |
| 20W4 | 8.4 | 79.5 | 3.4 | 3.3 | 71.6 |

Stored with 0% Enzyme Initially + 100% Enzyme at Start of Conversion

| | | | | | |
|---|---|---|---|---|---|
| CW1 | 9.2 | 86.4 | 3.7 | 3.6 | 78.5 |

### Example 3

### Washed Pretreated Softwood Cellulosic Cake 2-Week Storage with Enzyme

At T=2 weeks of storage, more enzymes were added to the flasks with 0.028 and 0.0 gram enzyme product/dry gram of pulp materials so that the total enzyme dose was 0.14 gram enzyme product/dry gram of pulp materials. Under the same testing conditions, pulp materials were hydrolyzed in a 50 mmol sodium citrate buffer (pH 4.8) at 50° C. and 200 rpm. After about 2 days of enzymatic hydrolysis, yeast seed was added to each flask at about 2 g/L for ethanol fermentation at 38° C. and at 100 rpm shaking speed for mixing. The final pulp consistency in fermentation was 15.7%.

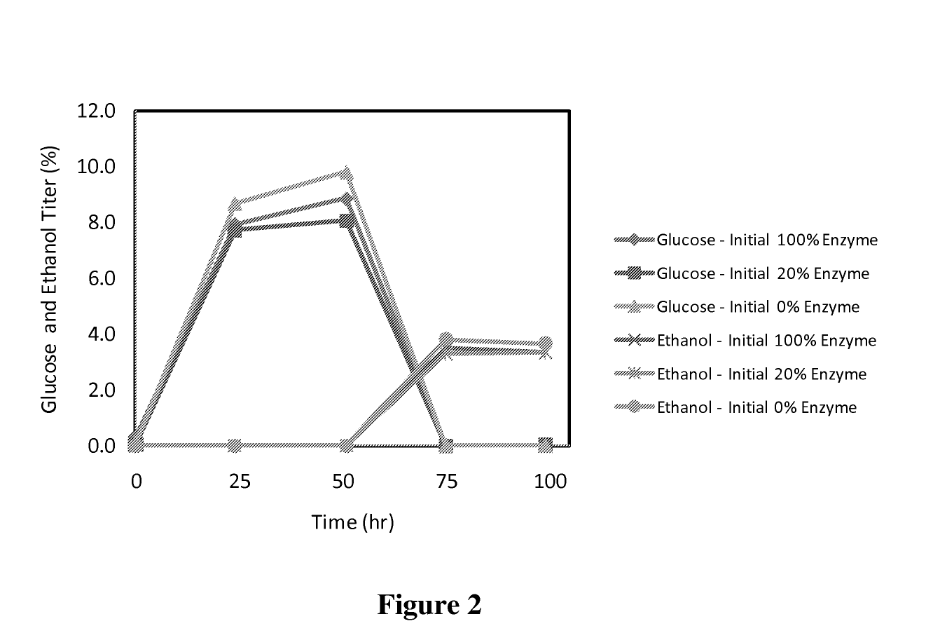

FIG. 2 shows the plot of glucose titers and ethanol concentrations during the course of hydrolysis and fermentation. Similar hydrolysis and fermentation trends were observed as in FIG. 1. At about 24 hours, most of the hydrolysis was observed to be completed. At 51 hours, yeast seed was added, after which ethanol fermentation was observed to be mostly completed in 24 hours. The actual time for hydrolysis and fermentation was observed to be as short as 48 hours.

The glucose yields of enzymatic hydrolysis at 51 hrs and ethanol titers at 75 and 99 hrs for the week 2 stored pulp cakes were determined, as shown below in Table 3. Test results show that most of the glucan was hydrolyzed at glucose yields of about 84% and about 76%, respectively for the initial 100% enzyme added pulp cake tests and for the initial 20% enzyme added pulp cake tests. The ethanol fermentation yields to total pulp sugar was about 74% and 72%, respectively for the initial 100% and 20% enzyme added cake tests. The control test had about 92% glucose yield and about 81% ethanol fermentation yield.

TABLE 3
Glucose yields and ethanol fermentation yields in pulp hydrolysis and
fermentation after T = 2 week pulp cake incubation with enzyme

| | | | | | | |
|---|---|---|---|---|---|---|
| | | | Normalized | | | Maximum |
| | | | Glucose | Ethanol | Ethanol | Ethanol |
| | | Glucose | Yield (%) | Titer | Titer | Yield (%) |
| | | (%) at | on Pulp | (%) at | (%) at | on Pulp |
| Week 2 | Test No. | 51 hr | Glucose | 75 hr | 99 hr | Sugar |

Pulp Cake Stored with 100% Enzyme Initially

| | | | | | |
|---|---|---|---|---|---|
| 100W5 | 9.0 | 85.3 | 3.5 | 3.3 | 74.5 |
| 100W6 | 8.7 | 81.7 | 3.5 | 3.4 | 74.0 |

Pulp Cake Stored with 20% Enzyme Initially + 80% Enzyme at Start of Conversion

| | | | | | |
|---|---|---|---|---|---|
| 20W5 | 8.2 | 76.9 | 3.4 | 3.4 | 72.0 |
| 20W6 | 8.0 | 75.0 | 3.2 | 3.3 | 71.2 |

Pulp Cake Stored with 0% Enzyme Initially + 100% Enzyme at Start of Conversion

| | | | | | |
|---|---|---|---|---|---|
| CW2 | 9.8 | 92.4 | 3.8 | 3.7 | 81.4 |

### Example 4

### Washed Pretreated Softwood Cellulosic Cake 4-Week Storage with Enzyme

At T=4 week storage, more enzymes were added to the flasks with 0.028 and 0.0 gram enzyme product/dry gram of pulp materials so that the total enzyme dose was 0.14 gram enzyme product/dry gram of pulp materials. Under the same testing conditions, pulp materials were hydrolyzed in a 50 mmol sodium citrate buffer (pH 4.8) at 50° C. and 200 rpm. After about two days of enzymatic hydrolysis, yeast seed was added to each flask at about 2 g/L for ethanol fermentation at 38° C. and at 100 rpm shaking speed for mixing. The final pulp consistency in fermentation was 15.7%.

The glucose yields of enzymatic hydrolysis at 51 hrs and ethanol titers at 75 and 99 hrs for the week 2 stored pulp cakes were determined, as shown below in Table 4. Test results show that most of the glucan was hydrolyzed at about 2 days. Subsequently, most of the ethanol fermentation was completed in the next 24 hours. These results showed that enzyme added to pulp cake materials for storage at ambient temperature could be a viable method to streamline the pretreated pulp storage, transportation and fermentation production.

TABLE 4
Glucose yields and ethanol fermentation yields in pulp hydrolysis and
fermentation after T = 4 week pulp cake incubation with enzyme

| | | | | | | |
|---|---|---|---|---|---|---|
| | | | Normalized | | | Maximum |
| | | | Glucose | Ethanol | Ethanol | Ethanol |
| | | Glucose | Yield (%) | Titer | Titer | Yield (%) |
| | | (%) at | on Pulp | (%) at | (%) at | on Pulp |
| Week 4 | Test No. | 51 hr | Glucose | 75 hr | 99 hr | Sugar |

Pulp Cake Stored with 100% Enzyme Initially

| | | | | | |
|---|---|---|---|---|---|
| 100W7 | 8.9 | 83.6 | 3.7 | 3.7 | 79.6 |
| 100W8 | 9.3 | 87.6 | 3.9 | 3.8 | 82.2 |

Pulp Cake Stored with 20% Enzyme Initially + 80% Enzyme at Start

| | | | | | |
|---|---|---|---|---|---|
| 20W7 | 10.6 | 100.4 | 4.2 | 3.9 | 90.1 |
| 20W8 | 10.7 | 100.9 | 4.2 | 3.9 | 90.4 |

Pulp Cake Stored with 0% Enzyme Initially + 100% Enzyme at Start

| | | | | | |
|---|---|---|---|---|---|
| CW3 | 10.6 | 100.0 | 4.3 | 4.0 | 91.0 |

### Example 5

### Unwashed Pretreated Softwood Cellulosic Cake Preparation and Storage with Enzyme at a Solid Content of 33%

The ground pretreated pulp had a pH of about 1.4. For safer material storage and transportation and for maintaining enzyme activity during pulp storage, the ground pulp materials were adjusted to above pH 4.0. In this test, the prehydrolysate or the cook liquor with pH 1.4 was first neutralized to pH 7.5 using calcium oxide. After autoclave, the pulp and the liquor were combined and mixed, and the final pH achieved about 5.0 without further addition of a base or an acid. The pH 5.0 pulp slurry was then pressed to filter out the excessive prehydrolysate, and the filtered pulp was formed into a cake on the filtration unit. The pressed pulp cake had a solid content of 33%, with a thickness of about 1 centimeter.

25 grams of the pulp cake were transferred into a 125-mL flat bottom Erlenmeyer flask. A spatula was used to tap the pulp cake tightly onto the flask bottom. The pulp cake in the flask was sterilized at 250° F. for 20 minutes. After cooling down to room temperature, Cellic® CTec2 cellulases at a dose of 0.13 gram enzyme product (nominal 100% enzyme)/dry gram of pulp materials was applied to one set of pulp cakes. The cellulases were only applied to the top of the pulp cake. The cellulases were evenly applied by a pipette, and no mixing was used. No cellulases were applied to the control set. Each flask mouth was then tightly wrapped with two layers of aluminum foil, and placed into a plastic tub wrapped with several layers of plastic wraps to avoid any moisture lost during storage. The tub with all the flasks was stored in an environmental chamber with a set temperature of 23° C. and a set humidity of 20%. Different sets of flasks were taken out after storage for enzymatic hydrolysis and fermentation.

At T=1 week storage, a set of the unwashed pulp cakes with 0.13 gram enzyme product/dry gram of pulp materials and a control set without previous enzyme addition were taken out. Enzymes were added to the control sets so that the total enzyme dose was 0.13 gram enzyme product/dry gram of pulp materials. After enzyme addition, a 50 mmol sodium citrate buffer (pH 4.8) was added to the pulp materials, and mixed using a spatula. The flasks were incubated in a shaking incubator at 50° C. and 200 rpm. After about 2 days of enzymatic hydrolysis, yeast seed was added to each flask at 2 g/L for ethanol fermentation. The fermentation temperature was controlled at 38° C. and the mixing was controlled at 100 rpm for flask mixing. The final pulp consistency in fermentation was 17.3%.

The glucose yields of the pulp hydrolysis and fermentation from the pulp cake stored for one week were determined, as shown below in Table 5. Results indicate that the stored pulp with 100% of the enzyme added initially increased the initial hydrolysis rate by 28.7% in the first 4 hours, suggesting that the total processing time for both hydrolysis and fermentation was surprisingly shortened. These results suggest that the enzyme addition to the stored pulp cake or block would reduce overall process time at the production site. Similar results were also observed for the tests after 2-week pulp cake storage, as shown below in Table 6. The 2-week pulp cake storage increased at least 38.8% initial glucan hydrolysis rate within 4.5 hrs. These results suggest that longer storage time with enzyme surprisingly increased enzymatic hydrolysis speed at the start of a formal hydrolysis and fermentation.

TABLE 5
Glucose yields and ethanol fermentation yields in pulp hydrolysis and
fermentation after T = 1 week unwashed pulp cake incubation with enzyme

| | | | | | | | |
|---|---|---|---|---|---|---|---|
| | | | | Normalized | | | Maximum |
| | | Glucose | Glucose | Glucose | Ethanol | Ethanol | Ethanol |
| | | (%) at 4.5 | (%) at 49 | Yield (%) on | Titer (%) | Titer (%) | Yield (%) on |
| Week 1 | Test No. | hr | hr | Pulp Glucose | at 78 hr | at 97 hr | Pulp Sugar |

Pulp Cake Stored with 100% Enzyme Initially

| | | | | | | |
|---|---|---|---|---|---|---|
| 1 | 6.7 | 10.5 | 103.7 | 4.7 | 4.6 | 90.3 |
| 2 | 6.8 | 10.4 | 103.4 | 4.6 | 4.8 | 92.3 |

Pulp Cake Stored with 0% Enzyme Initially + 100% Enzyme at Start of Conversion

| | | | | | | |
|---|---|---|---|---|---|---|
| 6 | 5.2 | 10.0 | 99.4 | 4.5 | 4.6 | 88.5 |
| 7 | 5.2 | 9.6 | 95.0 | 4.5 | 4.6 | 89.9 |

TABLE 6
Glucose yields and ethanol fermentation yields in pulp hydrolysis and
fermentation after T = 2 week unwashed pulp cake incubation with enzyme

| | | | | | | | |
|---|---|---|---|---|---|---|---|
| | | | | Normalized | | | Maximum |
| | | Glucose | Glucose | Glucose | Ethanol | Ethanol | Ethanol |
| | | (%) at 4.5 | (%) at 49 | Yield (%) on | Titer (%) | Titer (%) | Yield (%) on |
| Week 2 | Test No. | hr | hr | Pulp Glucose | at 78 hr | at 97 hr | Pulp Sugar |

Pulp Cake Stored with 100% Enzyme Initially

| | | | | | | |
|---|---|---|---|---|---|---|
| 3 | 7.7 | 9.8 | 96.9 | 4.7 | 4.5 | 91.2 |
| 4 | 7.7 | 9.9 | 97.5 | 4.5 | 4.5 | 86.0 |

Pulp Cake Stored with 0% Enzyme Initially + 100% Enzyme at Start of Conversion

| | | | | | | |
|---|---|---|---|---|---|---|
| 8 | 5.5 | 10.3 | 102.3 | 4.5 | 4.4 | 87.2 |
| 9 | 5.6 | 9.9 | 97.7 | 4.5 | 4.4 | 85.9 |

### Example 6

### Unwashed Pretreated Softwood Cellulosic Cake Preparation and Storage with Enzyme at a Solid Content of 44%

The ground pretreated pulp had a pH of about 1.4. For safer material storage and transportation and for maintaining enzyme activity during pulp storage, the ground pulp materials were adjusted to above pH 4.0. The prehydrolysate or the cook liquor with pH 1.4 was first neutralized to pH 7.5 using calcium oxide. After autoclave, the pulp and the liquor were combined and mixed. The final pH was about 5.0 without further addition of a base or an acid. The pH 5.0 pulp slurry was then pressed to filter out the excessive prehydrolysate. The filtered pulp was formed into a cake on the filtration unit. The pressed pulp cake had a solid content of 44%, and a thickness of about 1 centimeter.

19 grams of the pulp cake were transferred into a 125-mL flat bottom Erlenmeyer flask. A spatula was used to tap the pulp cake tightly onto the flask bottom. The pulp cake in the flask was sterilized at 250° F. for 20 minutes. After cooling down to room temperature, Cellic® CTec2 enzymes at a dose of 0.13 gram enzyme product (nominal 100% enzyme)/dry gram of pulp materials was applied to one set of pulp cakes. The enzymes were only applied to the top of the pulp cake. The enzymes were evenly applied by a pipette, and no mixing was used. No enzymes were applied to the control set. Each flask mouth was then wrapped tightly with two layers of aluminum foil, and placed into a plastic tub wrapped with several layers of plastic wraps to avoid any moisture lost during storage. The tub with all the flasks was stored in an environmental chamber with a set temperature of 23° C. and a set humidity of 20%. Different sets of flasks were taken out after storage for enzymatic hydrolysis and fermentation.

A set of the unwashed pulp cakes applied with 0.13 gram enzyme product/dry gram of pulp materials and a control set without previous enzyme addition were taken out. They were stored for one, two, and four weeks, respectively. The control without enzyme had a total of 0.13 gram enzyme product/dry gram of pulp materials added. After enzyme addition, a 50 mmol sodium citrate buffer (pH 4.8) was added to the pulp materials, and mixed using a spatula. The flasks were incubated in a shaking incubator at 50° C. and 200 rpm. After about 2 days of enzymatic hydrolysis, yeast seed was added to each flask at 2 g/L for ethanol fermentation. The fermentation temperature was controlled at 38° C. and the mixing was controlled at 100 rpm for flask mixing. The final pulp consistency in fermentation was 17.3%.

The glucose yields of the pulp hydrolysis and fermentation from the pulp cake stored for one, two and four weeks were determined, as shown below in Table 7. Results indicated that the stored pulp with 100% of the enzyme initially added increased the initial hydrolysis rate by 20% and 36% in the first four hours respectively for 2-week and 4-week stored pulp cake samples pre-added with enzymes. These results suggest that the total processing time for both hydrolysis and fermentation were shortened significantly. The longer storage time with enzyme surprisingly increased the rate of enzymatic hydrolysis at the start of a formal hydrolysis and fermentation process. These results suggest that the enzymes added and stored at a high solids content surprisingly maintained its activity, both in terms of rate of hydrolysis and overall yield, during the various storage periods.

TABLE 7
Glucose and ethanol fermentation yields in pulp hydrolysis and fermentation
after week 1, 2, and 4 unwashed pulp cake incubation with enzyme

| | | | | Normalized | |
|---|---|---|---|---|---|
| | | Initial Glucose | | Glucose | Maximum |
| | | Production Rate | | Yield (%) | Ethanol Yield |
| | Flask | Increase (%) vs. | Hydrolysis | on Pulp | (%) on Pulp |
| Conditions | No. | Control | (hrs) | Glucose | Sugar |
| Week 1 with | 1 | N/A* | 52 | 93.1 | 81.8 |
| 100% Enzyme | 2 | N/A | | 91.6 | 80.8 |
| Initially | | | | | |
| Week 1 Control | 7 | N/A | | 97.8 | 87.7 |
| with 0% Enzyme | 8 | N/A | | 95.6 | 85.2 |
| Initially + 100% | | | | | |
| Enzyme at Start | | | | | |
| of Conversion | | | | | |
| Week 2 with | 3 | 17.9 (4 hrs) | 52 | 95.3 | 78.6 |
| 100% Enzyme | 4 | 21.3 (4 hrs) | | 95.6 | 78.8 |
| Initially | | | | | |
| Week 2 Control | 9 | Control | | 100.5 | 87.3 |
| with 0% Enzyme | 10 | Control | | 99.5 | 85.8 |
| Initially + 100% | | | | | |
| Enzyme at Start | | | | | |
| of Conversion | | | | | |
| Week 4 with | 5 | 34.1 (4 hrs) | 24 | 93.1 | 78.9 |
| 100% Enzyme | 6 | 33.6 (4 hrs) | | 101.3 | 84.6 |
| Initially | | | | | |
| Week 4 Control | 11 | Control | | 101.6 | 86.6 |
| with 0% Enzyme | 12 | Control | | 102.9 | 87.0 |
| Initially + 100% | | | | | |
| Enzyme at Start | | | | | |
| of Conversion | | | | | |

*N/A means data not available.

### Example 7

### Unwashed Pretreated Hardwood Cellulosic Cake Preparation and Storage with Enzyme at a Solid Content of 45% and a Temperature of 40° C.

Maple hardwood chips were first pretreated. The resized wood chips were preheated in the digester, loaded with 12.5% calcium bisulfite on wood, and pretreated in a single-step temperature schedule: ramped from 90° C. to 155° C. in 15 minutes and held at 155° C. for 120 minutes. After cooking, the liquor was drained and the cooked chips were collected. The cooked chips were then sent to an Alpine grinder, without any water, to refine the chips into a pulp. The pulp batch number for this cook was CS10220A. This pulp was used in the following unwashed hardwood pulp tests.

The ground pretreated pulp had a pH of about 1.4. For safer material storage and transportation and for maintaining enzyme activity during pulp storage, the ground pulp materials were adjusted to above pH 4.0. The prehydrolysate or the cook liquor with pH 1.4 was first neutralized to pH 7.5 using calcium oxide. After autoclave, the pulp and the liquor were combined and mixed. The final pH was about 5.0 without further addition of a base or an acid. The pH 5.0 pulp slurry was then pressed to filter out the excessive prehydrolysate. The filtered pulp was formed into a cake on the filtration unit. The pressed pulp cake had a solid content of 45.2%, with a thickness of about 1 centimeter.

18 grams of the pulp cake were transferred into a 125-mL flat bottom Erlenmeyer flask. A spatula was used to tap the pulp cake tightly onto the flask bottom. The pulp cake in the flask was sterilized at 250° F. for 20 minutes. After cooling down to room temperature, Cellic® CTec2 enzyme at a dose of 0.16 gram enzyme product (nominal 100% enzyme)/dry gram of pulp materials was applied to one set of pulp cakes. The enzymes were only applied to the top of the pulp cake. The enzymes were evenly applied by a pipette, and no mixing was used. No enzymes were applied to the control set. After this procedure, each flask mouth was wrapped tightly with two layers of aluminum foil and placed into a plastic tub that was wrapped with several layers of plastic wraps to avoid any moisture lost during storage. The tub with all the flasks was stored in an environmental chamber with a set temperature of 40° C. Different sets of flasks were taken out after storage for enzymatic hydrolysis and fermentation.

A set of the unwashed pulp cakes applied with 0.16 gram enzyme product/dry gram of pulp materials and a control set without previous enzyme addition were taken out. They were storage for one, two, and four weeks, respectively. Enzymes were added to the control sets so that the total enzyme dose was 0.16 gram enzyme product/dry gram of pulp materials. After enzyme addition, a 50 mmol sodium citrate buffer (pH 4.8) was added to the pulp materials, and mixed using a spatula. The flasks were incubated in a shaking incubator at 50° C. and 200 rpm. After about 2 days of enzymatic hydrolysis, yeast seed was added to each flask at 2 g/L for ethanol fermentation. The fermentation temperature was controlled at 38° C., and the mixing was controlled at 100 rpm for flask mixing. The final pulp consistency in fermentation was 17.3%.

The glucose yields of the pulp hydrolysis and fermentation from the pulp cake stored for one, two and four weeks were determined, as shown below in Table 8. Results indicated that the stored pulp with 100% of the enzyme added initially increased the initial hydrolysis rate by 33% and 31% in the first 5 hours respectively for 1-week and 4-week stored pulp cake samples pre-added with enzymes. These results suggest that the total processing time for both hydrolysis and fermentation could be shortened, and that longer storage time with enzyme surprisingly increased enzymatic hydrolysis speed at the start of a formal hydrolysis and fermentation process. After storing at 40° C., the results suggest that the enzyme added during storage maintained its activity during the storage periods of week 1, week 2, and week 4 when compared to the controls.

TABLE 8
Glucose yields and ethanol fermentation yields in pulp hydrolysis and fermentation after
week 1, 2, and 4 unwashed pulp cake incubation with enzyme at 40° C.

| | | | | Normalized | |
|---|---|---|---|---|---|
| | | Initial Glucose | | Glucose | Maximum |
| | | Production Rate | | Yield (%) | Ethanol Yield |
| | Flask | Increase (%) vs. | Hydrolysis | on Pulp | (%) on Pulp |
| Conditions | No. | Control | (hrs) | Glucose | Sugar |
| Control | 1 | N/A | 51 | 100 | 83.6 |
| | 2 | N/A | | 100 | 80.9 |
| Week 1 with | 3 | 33.4 (5 hrs) | 51 | 97.8 | 78.7 |
| 100% Enzyme | | | | | |
| Initially | | | | | |
| Week 1 Control | 7 | Control | 51 | 101.8 | 80.7 |
| with 0% Enzyme | 8 | Control | | 101.9 | 81.2 |
| Initially + 100% | | | | | |
| Enzyme at Start | | | | | |
| of Conversion | | | | | |
| Week 2 with | 4 | 13.8 (19 hrs) | 51 | 92.7 | 77.7 |
| 100% Enzyme | | | | | |
| Initially | | | | | |
| Week 2 Control | 9 | Control | 51 | 86.0 | 83.0 |
| with 0% Enzyme | 10 | Control | | 86.8 | 78.9 |
| Initially + 100% | | | | | |
| Enzyme at Start | | | | | |
| of Conversion | | | | | |
| Week 4 with | 5 | 32.4 (5 hrs) | 51 | 89.9 | 77.6 |
| 100% Enzyme | 6 | 30.7 (5 hrs) | | 91.4 | 79.5 |
| Initially | | | | | |
| Week 4 Control | 11 | Control | 51 | 97.7 | 84.9 |
| with 0% Enzyme | 12 | Control | | 97.6 | 82.9 |
| Initially + 100% | | | | | |
| Enzyme at Start | | | | | |
| of Conversion | | | | | |

### Example 8

### Unwashed Pretreated Hardwood Cellulosic Cake Preparation and Storage with Enzyme at a Solid Content of 45% and Temperatures of 4° C. and −20° C.

The pretreated hardwood pulp cake materials described in Example 7 were also used to test the enzyme-added pulp cake materials for storage at 4° C. and −20° C. After storage, the enzyme-added pulp cakes were taken out, and hydrolysis tests were conducted at 50° C. and at 200 rpm, following the procedures described in Example 7. The yields of the pulp hydrolysis and fermentation from the pulp cake stored for one, two and four weeks were determined, as shown below in Table 9. At 4° C. and −20° C., the pulp cake with 100% of the enzyme initially added showed no significant increases in its initial hydrolysis rate. The enzymes, however, surprisingly remained active after storage at 4° C. and −20° C. These stored samples showed comparable glucose yields as compared to the controls. Results showed that the ethanol fermentation had high yields similar to the controls.

TABLE 9
Glucose yields and ethanol fermentation yields in pulp hydrolysis and fermentation after
week 1, 2, and 4, in which unwashed pulp cake was incubated with enzyme at 4° C.
and −20° C.

| | | | | Normalized | Maximum |
|---|---|---|---|---|---|
| | | | | Glucose | Ethanol |
| | Storage | | | Yield (%) | Yield (%) |
| | Temperature | Flask | Hydrolysis | on Pulp | on Pulp |
| Conditions | (° C.) | No. | (hrs) | Glucose | Sugar |
| Week 1 with 100% Enzyme | 4 | 1 | 24 | 90.7 | 81.8% |
| Initially | −20 | 2 | | 92.9 | 80.9% |
| Week 1 Control with 0% | 4 | 7 | 24 | 91.0 | 81.1% |
| Enzyme Initially + 100% | −20 | 8 | | 89.4 | 82.2% |
| Enzyme at Start of Conversion | | | | | |
| Week 2 with 100% Enzyme | 4 | 3 | 48 | 100.2 | 85.1% |
| Initially | −20 | 4 | | 101.2 | 87.2% |
| Week 2 Control with 0% | 4 | 9 | 48 | 101.4 | 88.3% |
| Enzyme Initially + 100% | −20 | 10 | | 99.2 | 88.7% |
| Enzyme at Start of Conversion | | | | | |
| Week 4 with 100% Enzyme | 4 | 5 | 48 | 96.0 | 75.1% |
| Initially | −20 | 6 | | 97.3 | 74.2% |
| Week 4 Control with 0% | 4 | 11 | 48 | 97.4 | 74.8% |
| Enzyme Initially + 100% | −20 | 12 | | 95.7 | 73.3% |
| Enzyme at Start of Conversion | | | | | |

### Example 9

### Unwashed Pretreated Switchgrass Cellulosic Cake Preparation and Storage with Enzyme at a Solid Content of 48% and a Temperature of 23° C.

Herbaceous biomass switchgrass materials were first pretreated. The resized switchgrass was preheated in the digester, loaded with 17.0% calcium bisulfite on dry biomass, and pretreated in a single-step temperature schedule: ramped from 90° C. to 155° C. in 15 minutes and held at 155° C. for 75 minutes. After cooking, the liquor was drained and the cooked switchgrass materials were collected. No further refining was needed since the pretreated switchgrass became a fine pulp after the pretreatment. The pulp batch number for this cook was CS10225A. This pulp was used in the following unwashed hardwood pulp tests.

The pretreated switchgrass pulp had a pH of about 1.4. For safer material storage and transportation and for maintaining enzyme activity during pulp storage, the ground pulp materials were adjusted to above pH 4.0. The prehydrolysate or the cook liquor with pH 1.4 was first neutralized to about pH 7.5 using calcium oxide. After the switchgrass pulp was mixed with the pH 7.5 (or above) switchgrass liquor, the pulp slurry pH was further adjusted to pH 5.3 by calcium oxide. The pH 5.3 pulp slurry was then filtered in a vacuum filtration unit and the excessive prehydrolysate was further pressed in a pneumatic pulp presser. The pressed pulp cake had a solid content of 48.0%, with a thickness of about 1 centimeter.

17.4 grams of the pulp cake were transferred into a 125-mL flat bottom Erlenmeyer flask. A spatula was used to tap the pulp cake tightly onto the flask bottom. The pulp cake in the flask was sterilized at 250° F. for 20 minutes. After cooling down to room temperature, Cellic® CTec2 enzyme at a dose of 0.13 gram enzyme product (nominal 100% enzyme)/dry gram of pulp materials was applied to one set of pulp cakes. The enzymes were only applied to the top of the pulp cake. The enzymes were evenly applied by a pipette, and no mixing was used. No enzymes were applied to the control set. After this procedure, each flask mouth was wrapped tightly with two layers of aluminum foil and placed into a plastic tub that was sealed into a plastic bag to avoid any moisture lost during storage. The tub with all the flasks was stored in an environmental chamber with a set temperature of 23° C. Different sets of flasks were taken out after storage for enzymatic hydrolysis and fermentation.

A set of the unwashed pulp cakes applied with 0.13 gram enzyme product/dry gram of pulp materials and a control set without previous enzyme addition were taken out. They were storage for one, two, and four weeks, respectively. Enzymes were added to the control sets so that the total enzyme dose was 0.13 gram enzyme product/dry gram of pulp materials. After enzyme addition, a 50 mmol sodium citrate buffer (pH 5.3) was added to the pulp materials, and mixed using a spatula. The flasks were incubated in a shaking incubator at 50° C. and 200 rpm. After about 2 days of enzymatic hydrolysis, yeast seed was added to each flask at 2 g/L for ethanol fermentation. The fermentation temperature was controlled at 38° C., and the mixing was controlled at 100 rpm for flask mixing. The final pulp consistency in fermentation was 17.3%.

The glucose yields of the pulp hydrolysis and fermentation from the pulp cake stored for one, two and four weeks were determined, as shown below in Table 10. Results indicated that the stored pulp with 100% of the enzyme added initially increased the average initial hydrolysis rate by 15%, 26% and 52% in the first 4-5 hours respectively for 1-week, 2-week and 4-week stored pulp cake samples pre-added with enzymes. These results suggest that the total processing time for both hydrolysis and fermentation could be shortened, and that longer storage time with enzyme surprisingly increased enzymatic hydrolysis speed at the start of a formal hydrolysis and fermentation process. After storing at 23° C., the results suggest that the enzyme added during storage maintained its activity during the storage periods of week 1, week 2, and week 4 when compared to the controls.

TABLE 10
Glucose yields and ethanol fermentation yields in pulp hydrolysis and fermentation after
week 1, 2, and 4, in which unwashed pulp cake was incubated with enzyme at 23° C.

| | | | | Normalized | |
|---|---|---|---|---|---|
| | | | | Glucose | Maximum |
| | | Initial Glucose | | Yield (%) | Ethanol Yield |
| | Flask | Production Rate | Hydrolysis | on Pulp | (%) on Pulp |
| Conditions | No. | Increase (%) | (hrs) | Glucose | Sugar |
| Week 1 with | 1 | 14.3 (5 hrs) | 48 | 101.9 | 81.6 |
| 100% Enzyme | 2 | 16.3 (5 hrs) | | 99.8 | 82.2 |
| Initially | | | | | |
| Week 1 Control | 7 | Control | 48 | 99.8 | 81.2 |
| with 0% Enzyme | 8 | Control | | 100.6 | 82.4 |
| Initially + 100% | | | | | |
| Enzyme at Start | | | | | |
| of Conversion | | | | | |
| Week 2 with | 3 | 24.6 (5 hrs) | 48 | 94.8 | 77.9 |
| 100% Enzyme | 4 | 26.6 (5 hrs) | | 93.4 | 79.1 |
| Initially | | | | | |
| Week 2 Control | 9 | Control | 48 | 93.6 | 77.8 |
| with 0% Enzyme | 10 | Control | | 94.3 | 80.5 |
| Initially + 100% | | | | | |
| Enzyme at Start | | | | | |
| of Conversion | | | | | |
| Week 4 with | 5 | 50.2 (4 hrs) | 51 | 99.2 | 74.3 |
| 100% Enzyme | 6 | 54.4 (4 hrs) | | 99.6 | 73.6 |
| Initially | | | | | |
| Week 4 Control | 11 | Control | 51 | 100.3 | 73.3 |
| with 0% Enzyme | 12 | Control | | 98.3 | 73.9 |
| Initially + 100% | | | | | |
| Enzyme at Start | | | | | |
| of Conversion | | | | | |

### Example 10

### Unwashed Pretreated Switchgrass Cellulosic Cake Preparation and Storage with Enzyme at a Solid Content of 48% and a Temperature of 40° C.

The pretreated switchgrass pulp cake materials described in Example 9 were also used to test the enzyme-added pulp cake materials for storage at 40° C. After storage, the enzyme-added pulp cakes were taken out, and hydrolysis tests were conducted at 50° C. and 200 rpm, following the procedures described in Example 9. The glucose yields of the pulp hydrolysis and fermentation from the pulp cake stored for one, two and four weeks were determined, as shown below in Table 11. At 40° C., the pulp cake with 100% of the enzyme initially added showed significant increases in its initial hydrolysis rate. The enzymes remained active after storage at 40° C. These stored samples showed comparable glucan hydrolysis yields as compared to the controls. Similarly, results showed that the ethanol fermentation had comparable ethanol yields as the controls.

TABLE 11
Glucose yields and ethanol fermentation yields in pulp hydrolysis and fermentation after
week 1, 2, and 4, in which unwashed pulp cake was incubated with enzyme at 40° C.

| | | | | Normalized | |
|---|---|---|---|---|---|
| | | | | Glucose | Maximum |
| | | Initial Glucose | | Yield (%) | Ethanol Yield |
| | Flask | Production Rate | Hydrolysis | on Pulp | (%) on Pulp |
| Conditions | No. | Increase (%) | (hrs) | Glucose | Sugar |
| Week 1 with | 1 | 25.2 (5 hrs) | 48 | 98.5 | 81.7 |
| 100% Enzyme | | | | | |
| Initially | | | | | |
| Week 1 Control | 7 | Control | 48 | 96.3 | 79.2 |
| with 0% Enzyme | | | | | |
| Initially + 100% | | | | | |
| Enzyme at Start | | | | | |
| of Conversion | | | | | |
| Week 2 with | 3 | 45.9 (5 hrs) | 48 | 97.5 | 74.4 |
| 100% Enzyme | | | | | |
| Initially | | | | | |
| Week 2 Control | 9 | Control | 48 | 94.9 | 74.2 |
| with 0% Enzyme | | | | | |
| Initially + 100% | | | | | |
| Enzyme at Start | | | | | |
| of Conversion | | | | | |
| Week 4 with | 5 | 33.6 (5 hrs) | 48 | 86.1 | 75.5 |
| 100% Enzyme | | | | | |
| Initially | | | | | |
| Week 4 Control | 11 | Control | 48 | 92.9 | 85.9 |
| with 0% Enzyme | | | | | |
| Initially + 100% | | | | | |
| Enzyme at Start | | | | | |
| of Conversion | | | | | |

### Example 11

### Unwashed Pretreated Switchgrass Cellulosic Cake Preparation and Storage with Enzyme at a Solid Content of 45% and a Temperature of 4° C.

Herbaceous biomass switchgrass materials were first pretreated. The resized switchgrass was preheated in the digester, loaded with 18.4% calcium bisulfite on dry biomass, and pretreated in a single-step temperature schedule: ramped from 90° C. to 155° C. in 15 minutes and held at 155° C. for 90 minutes. After cooking, the liquor was drained and the cooked switchgrass materials were collected. No further refining was needed since the pretreated switchgrass became a fine pulp after the pretreatment. The pulp batch number for this cook was CS10226A. The pressed pulp cake was prepared following the procedures in Example 9. This pulp was used in the following unwashed hardwood pulp tests.

The pretreated switchgrass pulp cake materials described in Example 9 were also used to test the enzyme-added pulp cake materials for storage at 4° C. After storage, the enzyme-added pulp cakes were taken out, and hydrolysis tests were conducted at 50° C. and at 200 rpm, following the procedures described in Example 9. The glucose yields of the pulp hydrolysis and fermentation from the pulp cake stored for one, two and four weeks were determined, as shown below in Table 12. At 4° C., the pulp cake with 100% of the enzyme initially added showed 12.4% and 47.2% increases in its initial hydrolysis rate, respectively for 1-week and 4-week storages. The enzymes surprisingly remained active after storage at 4° C. These stored samples showed comparable glucan hydrolysis yields as compared to the controls. Similarly, results showed that the ethanol fermentation had comparable ethanol yields as the controls.

TABLE 12
Glucose yields and ethanol fermentation yields in pulp hydrolysis and fermentation after
week 1, 2, and 4, in which unwashed pulp cake was incubated with enzyme at 4° C.

| | | | | Normalized | |
|---|---|---|---|---|---|
| | | | | Glucose | Maximum |
| | | Initial Glucose | | Yield (%) | Ethanol Yield |
| | Flask | Production Rate | Hydrolysis | on Pulp | (%) on Pulp |
| Conditions | No. | Increase (%) | (hrs) | Glucose | Sugar |
| Week 1 with | 1 | 12.4 (4 hrs) | 48 | 91.0 | 75.2 |
| 100% Enzyme | | | | | |
| Initially | | | | | |
| Week 1 Control | 7 | Control | 48 | 99.3 | 77.8 |
| with 0% Enzyme | | | | | |
| Initially + 100% | | | | | |
| Enzyme at Start | | | | | |
| of Conversion | | | | | |
| Week 2 with | 3 | N/A | 48 | 92.0 | 79.4 |
| 100% Enzyme | | | | | |
| Initially | | | | | |
| Week 2 Control | 9 | N/A | 48 | 100.0 | 77.5 |
| with 0% Enzyme | | | | | |
| Initially + 100% | | | | | |
| Enzyme at Start | | | | | |
| of Conversion | | | | | |
| Week 4 with | 5 | 47.2 (4 hrs) | 48 | 88.0 | 80.1 |
| 100% Enzyme | | | | | |
| Initially | | | | | |
| Week 4 Control | 11 | Control | 48 | 93.5 | 80.7 |
| with 0% Enzyme | | | | | |
| Initially + 100% | | | | | |
| Enzyme at Start | | | | | |
| of Conversion | | | | | |

### Example 12

### Unwashed Pretreated Hardwood Hydrolysis to Sugar After Storage with Enzyme at Room Temperature

Hardwood pulp was first pretreated. The pH of the pretreated hardwood pulp and the pretreatment liquor was then adjusted to a pH of about 5.0 using calcium oxide. After pH adjustment, the pulp was recovered by filtrating and pressing. The pulp materials were autoclaved at 121° C. for 20 minutes prior to the next steps. All the procedures below were completed in the sterile manners using a biosafety cabinet and autoclaved utensils and glassware.

The pH 5.0 pulp was first mixed with an enzyme dosage of 0.123 g enzyme product/dry g of pretreated biomass at a solid loading of 15.7%. After the pulp was mixed with the enzyme product for 20 minutes at room temperature, the pulp slurry was filtered and pressed to remove the liquid portion. The pressed pulp containing enzyme had a solid content of 37.6%. The enzyme containing wet pulp cake materials of 7.5 g dry pulp solid for each test were sealed and incubated inside 125-ml Erlenmeyer flasks at 23° C. for 13 days.

The amount of enzyme bound to the pressed pulp materials was determined by the total protein nitrogen analysis method. Specifically, the filtrate after enzyme mixing and pulp pressing was analyzed for the total Kjeldahl nitrogen (TKN). The TKN number was multiplied by the nitrogen weight factor (e.g., 6.25% in this Example) to give the total estimated enzyme protein on a weight basis. The control filtrate was observed to have no enzymes. The difference observed between the test filtrate and the control filtrate was the unbound enzyme protein nitrogen. The total added enzyme protein minus the unbound enzyme protein in the filtrate gave the amount of bound enzyme in the pulp cake.

Table 13 below shows that the tested pulp cake materials had a bound enzyme of 0.063 g enzyme product/dry g. The filtrate had 12.84 g enzyme product/L of unbound enzyme, which could be supplemented with more fresh enzymes for use in the next cake preparation.

TABLE 13
Bound enzyme in the pressed pretreated hardwood pulp

| Items | Amount | Units |
|---|---|---|
| Pretreated unwashed Hardwood Pulp | 30.0 | (dry g) |
| Added CTec2 Enzyme | 3.68 | (g enzyme product) |
| | 0.123 | (g enzyme product/g |
| | | pulp) |
| Initial Enzyme Titer in Buffer before | 24.06 | (g enzyme product/L) |
| Mixing with Pulp | | |
| Enzyme in Pressed Filtrate | 1.80 | (g enzyme product) |
| Bound Enzyme in Pulp | 1.88 | (g enzyme product) |
| | 51% | (enzyme wt % bound to |
| | | pulp) |
| | 0.063 | (g enzyme product/g |
| | | pulp) |
| Left Enzyme Titer in Filtrate | 12.84 | (g enzyme product/L) |

After the 13-day incubation, buffer was added to the pulp in each flask to achieve a solid content of about 15.7% for enzymatic hydrolysis. The buffer contained 50 mmol of sodium citrate to control the hydrolysis pH to about 5.0 during the hydrolysis. The pH was also periodically checked, and the pH was readjusted as necessary.

For each test, the enzyme used was from the pre-added dose and no additional enzyme was added. The control test had an enzyme dosage of 0.224 g enzyme product/dry g of the tested materials to release the maximum hydrolysable monomeric sugars as a control sugar baseline. The hydrolysis was conducted at 50° C. at 200 rpm on an orbital shaker. Based on the data summarized in Table 14 below, the normalized hydrolysis yield (averaged over the two tests) was about 93.2%.

TABLE 14
Sugar yield in the hydrolysis of the pressed pretreated
hardwood pulp pre-incubated with enzyme

| | Enzyme Dosage | | | Normalized |
|---|---|---|---|---|
| | (g enzyme | Hydrolysis | Final Sugar | Sugar |
| Test No. | product/g dry pulp) | Time (hrs) | Titer (%) | Yield (%) |
| Maple 1 | 0.063 | 72 | 11.4 | 92.5 |
| Maple 2 | 0.063 | 72 | 11.5 | 93.8 |
| Control | 0.224 | 72 | 12.2 | 100 (norm) |

### Example 13

### Washed Pretreated Softwood Hydrolysis to Sugar After Storage with Enzyme at Room Temperature

Softwood pulp was first pretreated. The pretreated softwood pulp was then mixed with water as a pre-washing process, and the pH was adjusted to about 5.0 using calcium oxide. After pH adjustment, the pulp was recovered by filtrating and pressing. The pulp materials were autoclaved at 121° C. for 20 minutes prior to the next steps. All the procedures below were completed in the sterile manners using a biosafety cabinet and autoclaved utensils and glassware. The pH 5.0 pulp was first mixed with an enzyme dosage of 0.099 g enzyme product/dry g of pretreated biomass at a solid loading of 15.7%. After the pulp was mixed with the enzyme product for 20 minutes at room temperature, the pulp slurry was filtered and pressed to remove the liquid portion. The pressed pulp containing enzyme had a solid content of 39.6%. The enzyme containing wet pulp cake materials of 7.5 g dry pulp solid for each test were sealed and incubated inside 125-ml Erlenmeyer flasks at 23° C. for 13 days.

The amount of enzyme bound to the pressed pulp materials was determined by the total protein nitrogen analysis method, as described in Example 12. Table 15 below shows that the tested pulp cake materials had 0.066 g enzyme product/dry g of the bound enzyme. The filtrate had 7.10 g enzyme product/L of unbound enzyme, which could be supplemented with fresher enzyme for use in the next cake preparation.

TABLE 15
Bound enzyme in the washed and pressed pretreated softwood pulp

| Items | Amount | Units |
|---|---|---|
| Pretreated unwashed Hardwood Pulp | 30.0 | (dry g) |
| Added CTec2 Enzyme | 3.68 | (g enzyme product) |
| | 0.099 | (g enzyme product/g |
| | | pulp) |
| Initial Enzyme Titer in Buffer before | 19.52 | (g enzyme product/L) |
| Mixing with Pulp | | |
| Enzyme in Pressed Filtrate | 1.01 | (g enzyme product) |
| Bound Enzyme in Pulp | 1.97 | (g enzyme product) |
| | 66% | (enzyme wt % bound to |
| | | pulp) |
| | 0.066 | (g enzyme product/g |
| | | pulp) |
| Left Enzyme Titer in Filtrate | 7.10 | (g enzyme product/L) |

After the 13-day incubation, buffer was added to the pulp in each flask to achieve a solid content of 15.7% for enzymatic hydrolysis. The buffer contained 50 mmol of sodium citrate to control the hydrolysis pH to about 5.0 during the hydrolysis. The pH was also periodically checked, and the pH was readjusted as necessary.

For each test, the enzyme used was from the pre-added dose and no additional enzyme was added. The control test had an enzyme dosage of 0.196 g enzyme product/dry g of the tested materials to release the maximum hydrolysable monomeric sugars as a control sugar baseline. The hydrolysis was conducted at 50° C. at 200 rpm on an orbital shaker. Based on the data summarized in Table 14 below, the normalized hydrolysis yield (averaged over the two tests) was about 90.4%.

TABLE 16
Sugar yield in the hydrolysis of the pressed pretreated
softwood pulp pre-incubated with enzyme

| | Enzyme Dosage | | Final | Normalized |
|---|---|---|---|---|
| | (g enzyme | Hydrolysis | Sugar | Sugar |
| Test No. | product/g dry pulp) | Time (hrs) | Titer (%) | Yield (%) |
| Softwood 1 | 0.066 | 72 | 7.8 | 91.9 |
| Softwood 2 | 0.066 | 72 | 7.7 | 88.9 |
| Control | 0.196 | 72 | 8.4 | 100 (norm) |

### Example 14

### Process with Washed Pulp Cake and its Application Without Enzyme Addition to Pulp Cake

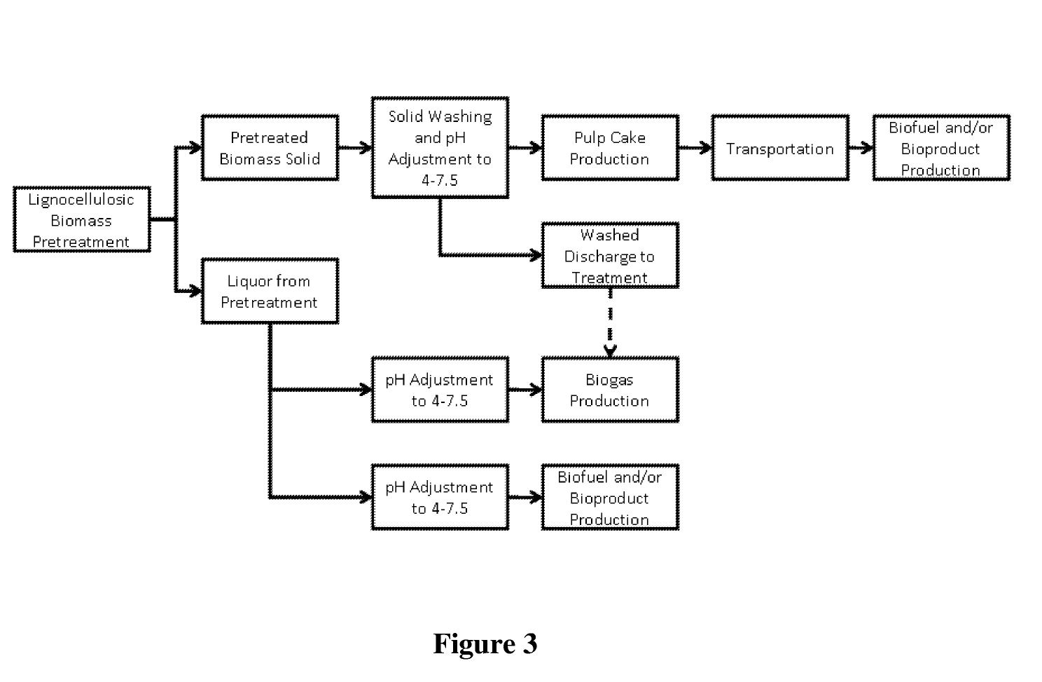

In the situation where the pretreated biomass plant is in one location and the fermentation plant for biofuel or bioproduct is in another location, a process could be designed for the production of pulp cake that could be packaged into a pulp slab, block, or roll for transportation. As illustrated in FIG. 3, the lignocellulosic biomass materials are pretreated in a pretreatment method including but not limited to green liquor, dilute acid, sulfite or bisulfite pulping, kraft pulping, hot water extraction, steam explosion with or without SO₂, and AFEX. After the pretreatment, the chip materials are separated away from the pretreated biomass solid, and the solid is washed and adjusted pH to 4-6. The washing effluent is sent to a wastewater treatment process for treatment and/or for biogas production and/or to an evaporator and boiler. The liquor stream containing higher concentration of hemicellulose sugars could also be used for biofuel or bioproduct production.

The washed pulp is filter-pressed or compressed to form a pulp cake that is subsequently stacked into a pulp slab or block or pellets, after clean-in-place packaging, ready for shipment or transportation to a biofuel or bioproduct plant. These pulp slab or block or rolls or pellets could also be stored in a storage facility before shipment or application. Before fermentation, the pulp cake will be diluted to a proper solid content and mixed with enzymes for enzymatic pulp hydrolysis.

### Example 15

### Process with Washed Pulp Cake and its Application with Enzyme Addition to Pulp Cake

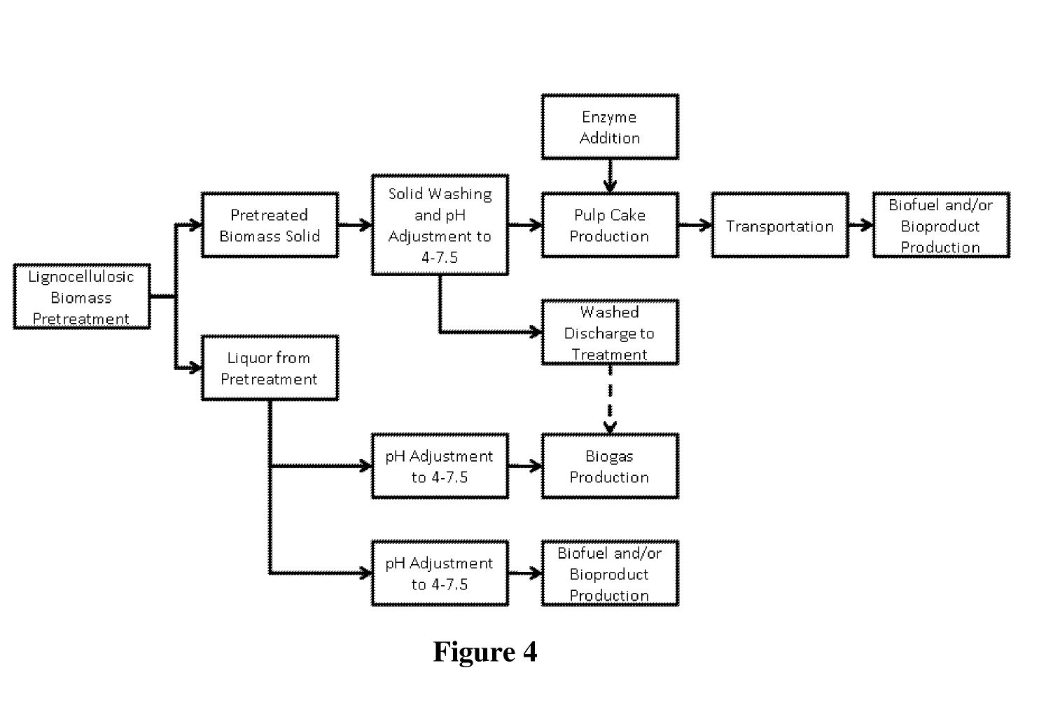

In the washed pulp cake process with enzyme addition, as illustrated in FIG. 4, after pH adjustment to 4-6, the washed pulp is filter-pressed or compressed to form a pulp cake or a pulp sheet, on top of which cellulolytic enzymes or cellulases are evenly sprayed at a proper enzyme dosage to pulp biomass. The enzyme containing pulp cake or sheet is subsequently stacked to form a pulp slab, block, roll or pellet, after clean packaging, ready for shipment or transportation to a biofuel or bioproduct plant. These pulp slab or block or pellets could also be stored in a storage facility before shipment or application. Before fermentation, the pulp cake with pre-added 100% enzyme dose will be diluted to a proper solid content and no cellulolytic enzymes or cellulases are to be added for the enzymatic pulp hydrolysis.

### Example 16

### Process with Unwashed Pulp Cake and its Application Without Enzyme Addition to Pulp Cake

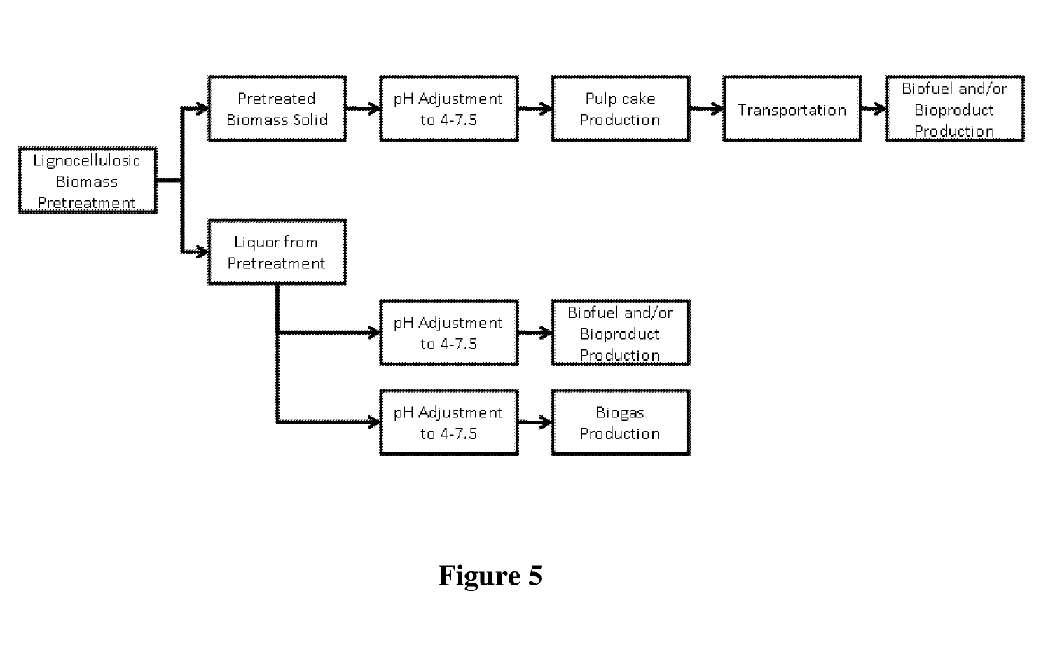

In the unwashed pulp cake process without enzyme addition, as seen in FIG. 5, after pH adjustment to 4-6, the unwashed pulp is filter-pressed or compressed to form a pulp cake that is subsequently stacked into a pulp slab or block or pellets, after clean-in-place packaging, ready for shipment or transportation to a biofuel or bioproduct plant. These pulp slabs, blocks, rolls or pellets could also be stored in a storage facility before shipment or application. Before fermentation, the pulp cake will be diluted to a proper solid content and mixed with cellulolytic enzymes or cellulases for enzymatic pulp hydrolysis.

### Example 17

### Process with Unwashed Pulp Cake and its Application with Enzyme Addition to Pulp Cake

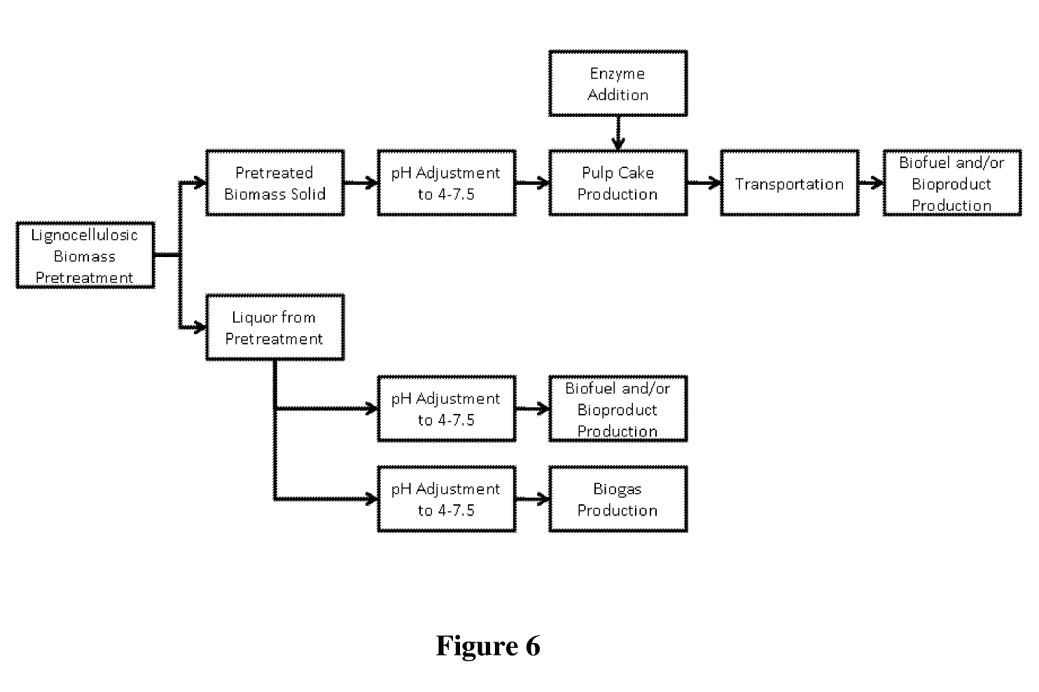

In the unwashed pulp cake process with enzyme addition, as seen in FIG. 6, after pH adjustment to 4-6, the washed pulp is filter-pressed or compressed to form a pulp cake or a pulp sheet, on top of which cellulolytic enzymes or cellulases are evenly sprayed at a proper enzyme dosage to pulp biomass. The enzyme containing pulp cake or sheet is subsequently stacked to form a pulp slab, block, roll or pellet, after clean packaging, ready for shipment or transportation to a biofuel or bioproduct plant. These pulp slab or block or pellets could also be stored in a storage facility before shipment or application. Before fermentation, the pulp cake with pre-added 100% enzyme dose will be diluted to a proper solid content and no cellulolytic enzymes or cellulases are to be added for the enzymatic pulp hydrolysis.

Although the methods described herein have been described in connection with some variations, it is not intended to be limited to the specific form set forth herein. Rather, the scope of the methods described herein is limited only by the claims. Additionally, although a feature may appear to be described in connection with particular variations, one skilled in the art would recognize that various features of the described variations may be combined in accordance with the methods described herein.

### Advantages of Applying Enzymes to Pretreated Lignocellulosic Biomass

The methods described herein present significant advantages over what is known in the art. In situations where a pretreatment facility is located on a different site than the conversion facility, there already exists a requirement for a delay to allow shipping of the pretreated biomass. This methods provided in this disclosure make productive use of that delay. Moreover, the methods provided in this disclosure produce predigested pretreated biomass that is ready for conversion at a production facility. This will enable, for example, corn-based ethanol plants to be upgraded from starch-based to cellulosic ethanol plants because pretreated, readily-hydrolyzable cellulose is available. Additionally, as bioconversion facilities become more widespread and come to depend on seasonally-available biomass sources, shipping of pretreated biomass is one way to mitigate swings in feedstock availability and reduce storage costs.

The methods provided herein confer significant cost savings to the conversion facilities receiving the pretreated lignocellulosic biomass. One such advantage is reduced capital expenditures. For example, a first generation ethanol plant can achieve cellulosic incentives without investing in pretreatment infrastructure. Since the pretreated lignocellulosic biomass is delivered with enzymes already applied, the first generation ethanol plant can avoid the need to build an enzyme production facility.

A second advantage is reduced operating expenses. For example, a cellulosic biofuel plant could have a reliable supplemental feedstock using the methods disclosed herein to offset the need for storage, especially of seasonally available feedstock. This can reduce the amount of enzymes applied for an unknown feedstock by 50% due to avoiding excessive safety margins, resulting in 20-30 cents/gal of benefit by having the enzymes added tailored specifically to the pretreated biomass. By avoiding the need for washing on site at the conversion facilities, water use and effluent volume could be reduced significantly compared to an integrated pretreatment, perhaps by as much as 80%.

A third advantage is reduced shipping of pretreated high density slabs compared to shipping biomass. Since the present disclosure provides methods that involve densifying the pretreated lignocellulosic biomass, conversion facilities could save as much as 3-4 times (as seen below in Table 17) the value density in shipping compared to untreated biomass, depending on the pretreatment chosen. Moreover, shipping a higher density material translates into a larger effective shipping distance, especially if rail is the shipping medium used.

TABLE 17
Shipping costs of pretreated high density slabs
compared to shipping biomass

| At 50% moisture and 12 lb/ft3 loose bulk density, and 65 | 0.39 gal/ft³ |
|---|---|
| gal/ODT, chips | |
| Density of wetlap slabs at 50% solids is 22.2 OD lb/ft3, | 1.443 gal/ft³ |
| at 149 gal/ODT (assuming kraft pulping as pretreatment) | |
| Ratio | 3.7 |

A fourth advantage to a centralized pretreatment method is feedstock security, wherein the conversion facilities have another source of feedstock that they cannot get locally, within a given radius. This advantage is highly relevant to a facility that depends on biomass that strives to operate at a full capacity.

The methods provided herein also confer significant savings to the pretreatment facilities. The methods avoid the energy cost of distillation, which amounts to about 20 lbs of steam per gallon of ethanol. This could be a 40-50% reduction in the steam demand for an integrated pretreatment/ethanol production plant.

What is claimed:

1. A method of producing pretreated biomass, the method comprising:
a) providing biomass;
b) applying a treatment method to the biomass to produce a pretreated biomass composition, wherein the pretreated biomass composition comprises a pretreatment liquor and pretreated biomass solids;
c) densifying the pretreated biomass solids to a solids content of 20% to 90% by removing liquid;
d) adding one or more hydrolysis enzymes to the pretreated biomass solids to form an enzyme-treated biomass; and
e) storing the enzyme-treated biomass.

2. The method of claim 1, further comprising adjusting the pH of the pretreated biomass solids to a pH range of 4.0 to 7.5.

3. The method of claim 1, wherein the biomass originates from softwood, hardwood, or an herbaceous plant.

4. The method of claim 1, wherein the enzyme-treated biomass is stored at a temperature between −30° C. to 50° C.

5. The method of claim 1, wherein the one or more hydrolysis enzymes are selected from the group consisting of cellulase, beta-glucosidase, xylanase, other hemicellulases, and mixtures thereof.

6. The method of claim 1, wherein the pretreated biomass solids are densified to form a pulp cake, sheet, roll, slab or block.

7. A method of producing pretreated biomass, the method comprising:
a) providing biomass;
b) applying a treatment method to the biomass to produce a pretreated biomass composition, wherein the pretreated biomass composition comprises a pretreatment liquor and pretreated biomass solids;
c) separating the pretreatment liquor from the pretreated biomass solids, wherein the pretreated biomass solids have a pH;
d) adjusting the pH of the pretreated biomass solids to a pH range of 4.0 to 7.5 to form a pH-adjusted pretreated biomass;
e) adding one or more hydrolysis enzymes to the pH-adjusted pretreated biomass solids to form an enzyme-treated biomass;
f) densifying the enzyme-treated biomass to a solids content of 20% to 90% by removing liquid to form a densified enzyme-treated biomass; and
g) storing the densified enzyme-treated biomass.

8. The method of claim 7, wherein the biomass originates from softwood, hardwood, or an herbaceous plant.

9. The method of claim 7, wherein the densified enzyme-treated biomass is stored at a temperature between −30° C. to 50° C.

10. The method of claim 7, wherein the one or more hydrolysis enzymes are selected from the group consisting of cellulase, beta-glucosidase, xylanase, other hemicellulases, and mixtures thereof.

11. The method of claim 7, wherein the densified enzyme-treated biomass is in the form of a pulp cake, sheet, roll, slab or block.

12. A method of producing pretreated biomass, the method comprising:
a) providing biomass;
b) applying a treatment method to the biomass to produce a pretreated biomass composition, wherein the pretreated biomass composition comprises a pretreatment liquor and pretreated biomass solids;
c) separating the pretreatment liquor from the pretreated biomass solids, wherein the pretreated biomass solids have a pH;
d) adjusting the pH of the pretreated biomass solids to a pH range of 4.0 to 7.5 to form pH-adjusted pretreated biomass solids;
e) densifying the pH-adjusted pretreated biomass solids by removing liquid to form a densified pretreated biomass, wherein the densified pretreated biomass has a solids content of 20% to 90%;
f) adding one or more hydrolysis enzymes to the densified pretreated biomass to form a densified enzyme-treated biomass; and
g) storing the densified enzyme-treated biomass.

13. The method of claim 12, wherein the biomass originates from softwood, hardwood, or an herbaceous plant.

14. The method of claim 12, wherein the densified enzyme-treated biomass is stored at a temperature between −30° C. to 50° C.

15. The method of claim 12, further comprising washing the pretreated biomass solids with water before step (d).

16. The method of claim 12, further comprising mixing the pretreated biomass solids with the pretreatment liquor before step (d).

17. The method of claim 12, further comprising adding one or more hydrolysis enzymes to the densified enzyme-treated biomass after step (f).

18. The method of claim 12, wherein the one or more hydrolysis enzymes are selected from the group consisting of cellulase, beta-glucosidase, xylanase, other hemicellulases, and mixtures thereof.

19. The method of claim 12, wherein the sugars produced by the hydrolysis are fermented with one or more fermentation organisms to produce a fermentation product.

20. The method of claim 12, wherein the densified pretreated biomass is in the form of a pulp cake, sheet, roll, slab or block.
